(19) United States
(12) Patent Application Publication
(10) Pub. No.: US 2018/0326021 A1
(43) Pub. Date: Nov. 15, 2018

(54) Methods and Compositions for the Treatment of Cytoplasmic Glycogen Storage Disorders

(71) Applicant: Duke University, Durham, NC (US)

(72) Inventors: Priya Kishnani, Durham, NC (US); Baodong Sun, Durham, NC (US); Dwight D. Koeberl, Durham, NC (US)

(21) Appl. No.: 15/755,912

(22) PCT Filed: Aug. 31, 2016
(86) PCT No.: PCT/US16/49680

§ 371 Date: Feb. 27, 2018

Related U.S. Application Data
(60) Provisional application No. 62/212,389, filed on Aug. 31, 2015.
(60) Provisional application No. 62/220,701, filed on Sep. 18, 2015.
(60) Provisional application No. 62/244,399, filed on Oct. 21, 2015.
(60) Provisional application No. 62/295,931, filed on Feb. 16, 2016.
(60) Provisional application No. 62/331,225, filed on May 3, 2016.
(60) Provisional application No. 62/331,166, filed on May 3, 2016.

(51) Int. Cl. A61K 38/47; A61K 45/06; A61P 3/00

(52) U.S. Cl. CPC A61K 38/47; A61P 3/00; A61K 45/06; C12Y 302/0102

(57) ABSTRACT

The present disclosure is directed to methods of treating a cytoplasmic glycogen storage disorder, including glycogen storage disease I, glycogen storage disease III, glycogen storage disease IV, and/or conditions associated with a PRKAG2 mutation, by administering a lysosomal enzyme such as acid alpha-glucosidase. Conditions associated with a PRKAG2 mutation may include hypotonia, cardiomyopathy, myopathy, cytoplasmic glycogen accumulation, ventricular hypertrophy, severe infantile hypertrophic cardiomyopathy, heart rhythm disturbances, increased left ventricular wall thickness, ventricular pre-excitation, or a combination thereof. Methods of treating a cytoplasmic glycogen storage disorder by administering a lysosomal enzyme and a second therapeutic agent are also described. Other embodiments are directed to methods of treating a cytoplasmic glycogen storage disorder by administering a therapeutic agent as an adjunctive therapy to lysosomal enzyme replacement therapy.

### CROSS REFERENCE TO RELATED APPLICATIONS

This application claims the benefit of priority to U.S. Provisional Application No. 62/212,389 filed Aug. 31, 2015; U.S. Provisional Application No. 62/295,931 filed Feb. 16, 2016; U.S. Provisional Application No. 62/331,166 filed May 3, 2016; U.S. Provisional Application No. 62/244,399 filed Oct. 21, 2015; U.S. Provisional Application No. 62/331,225 filed May 3, 2016; and U.S. Provisional Application No. 62/220,701 filed Sep. 18, 2015, the disclosure of each of which is incorporated by reference herein in its entirety.

### SUMMARY

Embodiments herein are directed to treating a cytoplasmic glycogen storage disorder in an individual comprising administering to the individual a lysosomal enzyme (e.g. an acid alpha-glucosidase (acid α-glucosidase or GAA)). In some embodiments, the method further comprises administering a therapeutic agent in addition to the lysosomal enzyme. Some embodiments herein are directed to a method of treating a cytoplasmic glycogen storage disorder in an individual in need thereof comprising administering to the individual a therapeutic agent as an adjunctive therapy to a lysosomal enzyme.

In some embodiments, the lysosomal enzyme is selected from glucocerebrosidase, acid alpha-glucosidase, alpha-galactosidase, alpha-n-acetylgalactosaminidase, acid sphingomyelinase, alpha-iduronidase, or a combination thereof. In some embodiments, the lysosomal enzyme may be acid alpha-glucosidase. In some embodiments, the acid alpha-glucosidase may be selected from a GAA, recombinant human acid alpha-glucosidase (rhGAA), alglucosidase alfa, neo-rhGAA, reveglucosidase alpha, an rhGAA administered with a chaperone (e.g. 1-deoxynojirimycin (DNJ), α-homonojirimycin, or castanospermine), a chimeric polypeptide comprising any of the foregoing (e.g. a chimeric polypeptide of GAA and a 3E10 anitbody, or GAA tagged with a moiety that promotes transit via an equilibrative nucleoside transporter 2 (ENT2)), a portion thereof, or a combination thereof.

In some embodiments, the cytoplasmic glycogen storage disorder may be selected from glycogen storage disease type I (GSD I), glycogen storage disease III (GSD III), glycogen storage disease IV (GSD IV), glycogen storage disease V (GSD V), glycogen storage disease VI (GSD VI), glycogen storage disease VII (GSD VII), glycogen storage disease IX (GSD IX), glycogen storage disease XI (GSD XI), glycogen storage disease XII (GSD XII), glycogen storage disease XIII (GSD XIII), glycogen storage disease XIV (GSD XIV) (phosphoglucomutase deficiency), Danon disease (GSD 2B, LAMP-2 deficiency), Lafora disease, a condition associated with protein kinase gamma subunit 2-deficiency (PRKAG2) deficiency, any other condition where there is cytoplasmic accumulation of glycogen, or a combination thereof. In some embodiments, the cytoplasmic glycogen storage disorder may be GSD III, GSD IV or a condition associated with protein kinase gamma subunit 2-deficiency (PRKAG2) deficiency.

In some embodiments, the therapeutic agent may be selected from a growth hormone, an autocrine glycoprotein, a β2 agonist, an agent to treat or prevent hypoglycemia (e.g. cornstarch), an agent to treat or prevent hyperlipidemia (e.g. HMG-CoA; ACE inhibitors), an agent to treat or prevent neutropenia, an agent to suppress glycogen synthase (e.g. RNAi; 20(S)-protopanaxadiol), an agent to prevent or reverse glycogen synthesis, an agent to treat or prevent fibrosis, an agent to improve mitochondrial function, an agent to treat any other symptom, such as those described herein, of the cytoplasmic storage disorders of embodiments herein, or a combination thereof.

Some embodiments herein are directed to methods of treating a cytoplasmic glycogen storage disorder comprising administering a β2 agonist and an acid α-glucosidase to a subject in need thereof. In some embodiments, the β2 agonist is a selective β2 agonist. In some embodiments, the β2 agonist is albuterol, arbutamine, bambuterol, befunolol, bitolterol, bromoacetylalprenololmenthane, broxaterol, carbuterol, cimaterol, cirazoline, clenbuterol, clorprenaline, denopamine, dioxethedrine, dopexamine, ephedrine, epinephrine, etafedrine, ethylnorepinephrine, etilefrine, fenoterol, formoterol, hexoprenaline, higenamine, ibopamine, isoetharine, isoproterenol, isoxsuprine, mabuterol, metaproterenol, methoxyphenamine, norepinephrine, nylidrin, oxyfedrine, pirbuterol, prenalterol, procaterol, propranolol, protokylol, quinterenol, ractopamine, reproterol, rimiterol, ritodrine, salmefamol, soterenol, salmeterol, terbutaline, tretoquinol, tulobuterol, xamoterol, zilpaterol, zinterol, or a combination thereof. In some embodiments, the β2 agonist may be clenbuterol.

Some embodiments are directed to a method of treating GSD III in an individual in need thereof comprising administering to the individual a composition comprising a β2 agonist and an acid alpha-glucosidase. Some embodiments are directed to a method of treating GSD IV in an individual in need thereof comprising administering to the individual a composition comprising a β2 agonist and an acid alpha-glucosidase.

Some embodiments are directed to a method of treating cytoplasmic glycogen storage disorder in an individual in need thereof comprising administering to the individual a therapeutically effective amount of a lysosomal enzyme, wherein the lysosomal enzyme is administered at a first higher therapeutically effective dose weekly until a desired response is reached and then the lysosomal enzyme is administered at a second lower therapeutically effective dose at a regular interval. In some embodiments, the first higher therapeutically effective dose is about 40 mg/kg to about 100 mg/kg. In some embodiments, the second lower therapeutically effective dose is about 20 mg/kg to about 80 mg/kg. In some embodiments, the regular interval is selected from bimonthly, monthly, biweekly, weekly, twice weekly, daily, twice a day, three times a day, or more often a day.

In some embodiments, the method further comprises pretreating the individual with an immune modulator prior to administration of the lysosomal enzyme. In some embodiments, the individual being treated does not have a significant amount of fibrosis. In some embodiments, the individual being treated does not have a significant amount of fibrosis in the liver. In some embodiments, the individual being treated does not have a significant amount of fibrosis in the liver, skeletal muscle, heart, brain, or a combination thereof.

Some embodiments are directed to a method of treating glycogen storage disorder I (GSD I) in an individual in need thereof comprises administering to the individual a therapeutically effective amount of an acid alpha-glucosidase. In some embodiments, the GSD I is selected from GSD Ia, GSD Ib, GSD Ic, or a combination thereof. In some embodiments, the individual has steatosis. In some embodiments, the method further includes administration of an additional therapeutic agent. In some embodiments, the method further includes administration of an additional therapeutic agent that increases uptake of the acid alpha-glucosidase. In some embodiments, the additional therapeutic agent is a β2 agonist.

Some embodiments are directed to a method of treating a condition associated with PRKAG2 deficiency in an individual in need thereof comprises administering to the individual a therapeutically effective amount of an acid alpha-glucosidase. In some embodiments, the condition associated with PRKAG2 deficiency is selected from hypotonia, cardiac hypertrophy, cardiomyopathy, myopathy, cytoplasmic glycogen accumulation, ventricular hypertrophy, severe infantile hypertrophic cardiomyopathy, heart rhythm disturbances, increased left ventricular wall thickness, ventricular preexcitation, any other condition seen in patient having PRKAG2 deficiency, including glycogenosis or cardiac glycogenosis due to AMP-activated PRKAG2 deficiency, or a combination thereof. In some embodiments, the PRKAG2 deficiency is due to a mutation selected from PRKAG2 Het R531Qh mutation, PRKAG2 R302G mutation, PRKAG2 T400N mutation, PRKAG2 N4881 missense mutation, PRKAG2 R531G missense mutation, PRKAG2 G100S missense mutation, or a combination thereof.

Some embodiments are directed to a method of improving motor skills in an individual with a PRKAG2 gene mutation comprising administering to the individual a therapeutically effective amount of acid alpha-glucosidase. Some embodiments are directed to a method of improving muscle strength and function in an individual with a PRKAG2 gene mutation comprising administering to the individual a therapeutically effective amount of acid alpha-glucosidase. Some embodiments are directed to a method of decreasing seizures in an individual with a PRKAG2 gene mutation comprising administering to the individual a therapeutically effective amount of acid alpha-glucosidase.

Some embodiments are directed to a composition comprising a therapeutic agent of embodiments herein and a lysosomal enzyme of embodiments herein. Some embodiments are directed to a composition comprising a β2 agonist and an acid alpha-glucosidase.

### DESCRIPTION OF THE FIGURES

FIG. 1 illustrates the generation of heterozygous Agl^{+/-} mice by one-step cross-breeding Agl^{Tmla} mice with CMV-Cre mice.

FIG. 2 illustrates an analysis of the skeletal muscle biopsies from two GSD Ma patients, a 45 year old male (Pt. 1) and a 35 year old female (Pt. 2). (A) High-resolution light microscopy demonstrates that purple-staining glycogen is present as non-membrane-bounded cytoplasmic lakes within myocytes by Periodic Acid Schiff (PAS) staining (scale bar=20 μm). (B) Under EM, occasional lysosomal glycogen (arrow) was also seen in the myocytes (scale bar=1 μm). (C) Glycogen accumulation pattern revealed that glycogen content was peaked at Day 15 in cultured patient muscle cells. (D) Glucose starvation experiment showed incomplete glycogen utilization in the muscle cells from both GSD Ma patients compared to a normal control subject (Nor). (E) GAA activity in normal and patient cells 48 h after adding recombinant human acid alpha-glucosidase (rhGAA, Myozyme, alglucosidase alfa) treatment. (F) rhGAA significantly reduced glycogen concentration in both normal and patient cells. Mean±standard deviation is shown in C—F (n=4). The significance of differences between two different groups was assessed using the two-tailed, equal variance student T-test (*P<0.001; **P<0.01; ***P<0.05).

FIG. 3 illustrates the (A) GAA activity and (B) glycogen content in primary GSD IV mouse muscle cells with (rhGAA) or without (untreated, UT) rhGAA treatment. rhGAA treatment significantly (p<0.01) reduced glycogen content in these cells. Data were average of two independent experiments±SD.

FIG. 4 illustrates that rhGAA treatment reduced glycogen deposition in GSD IV mouse myoblasts. Glycogen was stained with an α-glycogen monoclonal antibody (ESG1A9mAb).

FIG. 5 illustrates progressive glycogen deposits in various muscles of GSD IV mice. There were no or very scarce PAS positive particles detected in muscles detected in muscles at 1 month of age. Significant amount of PAS positive cells were observed at 3 months and 6 months of age, indicating the progressive nature of glycogen accumulation in GSD IV.

FIG. 6 illustrates glycogen deposits in the diaphragm, heart, and brain of GSD IV mice. PAS positive particles were detected in these tissues at 3 months of age and became more prevalent at 6 months of age.

FIG. 7A illustrates the GBE enzyme activity and FIG. 7B illustrates the glycogen content in GSD IV mice and wild-type (WT) mice at age of 3 months. The percentage of residual GBE activity in the GSD IV mice to WT mice was shown in A. n=5.

FIG. 8 illustrates glycogen content in skeletal muscles from wild-type (Wt) and GSD animals. FIG. 8A illustrates representative PAS staining of muscle (gastrocnemius) sections form Wt mice, GSD II mice, GSD Ma dogs, and GSD IV mice (magnification 400×). FIG. 8B illustrates comparison of the STD-prep and the Boil-prep methods for quantitation of glycogen in muscles from animals in A. n=5 for mice, n=4 for dogs.

FIG. 9 illustrates measurement of glycogen content in other tissues from the GSD IV mice. FIG. 9A is a PAS staining which shows glycogen deposits of various degrees in liver, diaphragm, heart and brain (cerebrum) of the GSD IV mice (magnification 400×). FIG. 9B is a comparison of the STD-prep and the Boil-prep methods for quantitation of glycogen in these tissues. n=5 mice.

FIG. 10 illustrates a comparison of the STD-prep and the Boil-prep methods for quantitation of glycogen in cultured skin fibroblasts from a patient with GSD II and one with GSD IV. Average±standard deviation of n=4 plates for each patient are shown.

FIG. 11 illustrates (A) rhGAA uptake by tissues of GSD IV mice upon administration of 20 mg/kg, 40 mg/kg, or 100 mg/kg rhGAA; (B) clearance of glycogen accumulation in various tissues upon administration of 20 mg/kg, 40 mg/kg, or 100 mg/kg rhGAA; (C) measure of hepatomegaly (liver/body weight ratio) in GSD IV mice upon administration of 20 mg/kg or 40 mg/kg rhGAA; (D) levels of liver enzyme alanine transaminase (ALT) in GSD IV mice upon administration of 20 mg/kg or 40 mg/kg rhGAA; and (E) levels of liver enzyme aspartate transaminase (AST) in GSD IV mice upon administration of 20 mg/kg or 40 mg/kg rhGAA. rhGAA at indicated doses was intravenously injected into GSD IV mice once per weeks for 4 weeks.

FIG. 12 illustrates (A) enzyme uptake; and (B) clearance of glycogen in tissues of GSD III mice upon weekly intravenous administration of 20 mg/kg, 40 mg/kg, or 100 mg/kg rhGAA for 4 weeks.

FIG. 13 illustrates the effect of rhGAA treatment on ratio of liver/body weight of GSD III mice upon weekly upon weekly intravenous administration of 20 mg/kg, 40 mg/kg, or 100 mg/kg rhGAA for 4 weeks.

FIG. 14 illustrates the (A) plasma AST levels; (B) plasma ALT levels; (C) plasma ALP levels; and (D) plasma CK levels of GSD III mice upon weekly administration of 20 mg/kg, 40 mg/kg, or 100 mg/kg rhGAA for 4 weeks.

FIG. 15 illustrates the schematic mechanism of AMPK-mediated increase in cardiac and skeletal muscle glycogen accumulation in PRKAG2 deficiency. Mutations in the PRKAG2 gene, which encodes the regulatory γ2 subunit, cause chronic activation of AMPK. Elevated AMPK activity promotes glucose transporter 4 (GLUT4) shuttling to the plasma membrane and increases glucose uptake and intracellular glucose 6-phosphate (G6P) concentration. This leads to an allosteric activation of glycogen synthase (GS), which overrides the inhibitory effect of AMPK on GS, resulting in a net increase in GS activity and excess glycogen storage in muscle cells.

FIG. 16 illustrates a high resolution light microscopy of quadriceps muscle biopsy from a patient with PRKAG2 deficiency at age 44 months. Patient was not on ERT at the time of biopsy (off ERT for 11 months). One-micron semithin epon sections were stained with Richardsons/PAS stain combination. PAS positive blebs (arrow), are present at the periphery of some cells, suggestive of glycogen accumulation.

FIG. 17 illustrates electron microscopy of quadriceps muscle biopsy from a patient with PRKAG2 deficiency at age 44 months. Patient was not on ERT at the time of biopsy (off ERT for 11 months). The myofibrillar structure of the myocytes was largely intact in most fields. There were isolated foci of frayed and degenerated myofibrils interrupted by small pools of cytoplasmic glycogen.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “β2 agonist” is a reference to one or more β2 agonists and equivalents thereof known to those skilled in the art, and so forth.

As used herein, the term “about” means plus or minus 5% of the numerical value of the number with which it is being used. Therefore, about 50% means in the range of 45%-55%.

“Adjuvant” or “adjunctive” therapy, as used herein, refers to therapy that is given in addition to the primary, main, or initial therapy to maximize its effectiveness. For example, in some embodiments, herein a therapeutic agent, such as a β2 agonist, may be administered as an adjunctive therapy to a lysosomal enzyme, such as GAA, in order to increase uptake of the lysosomal enzyme. In some embodiments, the adjunctive therapy may be co-administered or sequentially administered.

“Administering”, when used in conjunction with a therapeutic, means to administer a therapeutic directly into or onto a target tissue or to administer a therapeutic to a subject, whereby the therapeutic positively impacts the tissue to which it is targeted. Thus, as used herein, the term “administering”, when used in conjunction with a therapeutic, can include, but is not limited to, providing a therapeutic to a subject systemically by, for example, intravenous injection, whereby the therapeutic reaches the target tissue. Administering a composition or therapeutic may be accomplished by, for example, injection, oral administration, topical administration, or by these methods in combination with other known techniques. Such combination techniques may include heating, radiation, ultrasound and the use of delivery agents. Preferably, administering is a self-administration, wherein the therapeutic or composition is administered by the subject themselves. Alternatively, administering may be administration to the subject by a health care provider.

The terms, “treat” and “treatment,” as used herein, refer to amelioration of one or more symptoms associated with the disease, prevention or delay of the onset of one or more symptoms of the disease, and/or lessening of the severity or frequency of one or more symptoms of the disease. For example, treatment can refer to improvement of hypoglycemia, growth retardation, hepatomegaly, and hepatic function (e.g., reduction of SGOT, SGPT); cardiac status (e.g., reduction, amelioration or prevention of the progressive cardiomyopathy, arrhythmia and other cardiac manifestations that can be found, for example, in GSD-III), myopathy (e.g., exercise tolerance), reduction of glycogen levels in tissue (e.g., liver and muscle) of the individual affected by the disease, or any combination of these effects. Further, the treatment may prevent long term complications, such as, liver cirrhosis and hepatocellular carcinoma due to clearance of glycogen with an abnormal structure, atherosclerosis secondary to hyperlipidemia, ventricular hypertrophy, and reduced bone mineral density. In some embodiments, treatment includes improvement in liver enzyme levels, improvement in glycogen levels, improvement of liver symptoms, particularly, in reduction or prevention of GSD (e.g., GSD-III)-associated hypoglycemia, hepatomegaly, abnormal liver function, liver inflammation, and cirrhosis.

The terms, “improve,” “prevent” or “reduce,” as used herein, indicate values that are relative to a baseline measurement, such as a measurement in the same individual prior to initiation of the treatment described herein, or a measurement in a control individual (or multiple control individuals) in the absence of the treatment described herein. A control individual is an individual afflicted with the same form of the disease (e.g., GSD-III) as the individual being treated, who is about the same age as the individual being treated (to ensure that the stages of the disease in the treated individual and the control individual(s) are comparable).

As used herein, the term “therapeutic agent” means an agent utilized to treat, combat, ameliorate, prevent or improve an unwanted condition or disease of a subject. In part, embodiments described herein may be directed to the treatment of various cytoplasmic glycogen storage disorders, including, but not limited to glycogen storage disease type I (GSD I), glycogen storage disease III (GSD III), glycogen storage disease IV (GSD IV), glycogen storage disease V (GSD V), glycogen storage disease VI (GSD VI), glycogen storage disease VII (GSD VII), glycogen storage disease IX (GSD IX), glycogen storage disease XI (GSD XI), glycogen storage disease XII (GSD XII), glycogen storage disease XIII (GSD XIII), glycogen storage disease XIV (GSD XIV) (phosphoglucomutase deficiency), Danon disease (GSD 2B, LAMP-2 deficiency), Lafora disease, glycogenosis due to AMP-activated protein kinase gamma subunit 2-deficiency (PRKAG2), or cardiac glycogenosis due to AMP-activated protein kinase gamma subunit 2 deficiency. In some embodiments, GSD-III may be selected from GSD-type IIIa, type IIIb, type IIIc, or type IIId.

The terms “therapeutically effective” or “effective”, as used herein, may be used interchangeably and refer to an amount of a therapeutic composition of embodiments described herein. For example, a therapeutically effective amount of a composition is an amount of the composition, and particularly the active ingredient, such as GAA, that generally achieves the desired effect. For example, the desired effect can be an improvement, prevention, or reduction of a particular disease state.

A “therapeutically effective amount” or “effective amount” of a composition is an amount necessary or sufficient to achieve the desired result or clinical outcome. For example, the desired result or clinical outcome can be an improvement, prevention, or reduction of a particular disease state. The therapeutic effect contemplated by the embodiments herein includes medically therapeutic, cosmetically therapeutic and/or prophylactic treatment, as appropriate. The specific dose of a compound administered according to embodiments described herein to obtain therapeutic effects will, of course, be determined by the particular circumstances surrounding the case, including, for example, the compound administered, the route of administration, and the condition being treated. However, the effective amount administered can be determined by the practitioner or manufacturer or patient in light of the relevant circumstances including the condition to be treated, the choice of compound to be administered, and the chosen route of administration, and therefore, the above dosage ranges are not intended to limit the scope of the invention in any way. A therapeutically effective amount of the compound of embodiments herein is typically an amount such that when it is administered in a physiologically tolerable excipient composition, it is sufficient to achieve an effective systemic concentration or local concentration in or on the tissue to achieve the desired therapeutic or clinical outcome.

As used herein, the term “comprising” or “comprises” means that the composition or method is broad in scope and may include, but does not necessarily include, elements, steps, or ingredients other than that specifically recited in the particular claimed embodiment or claim.

As used herein, the term “consists of” or “consisting of” means that the composition or method includes only the elements, steps, or ingredients specifically recited in the particular claimed embodiment or claim.

As used herein, the term “consisting essentially of” or “consists essentially of” means that the composition or method includes only the specified materials or steps and those that do not materially affect the basic and novel characteristics of the claimed invention.

Generally speaking, the term “tissue” refers to any aggregation of similarly specialized cells which are united in the performance of a particular function.

The individual, patient, or subject being treated may be a human (infant, child, adolescent, or adult human) having the disease to be treated, e.g. GSD IV. The individual may have residual enzyme (e.g. GBE) activity, or no measurable activity. In some embodiments, the individual may be an individual who has been recently diagnosed with the disease. Early treatment (treatment commencing as soon as possible after diagnosis) may be important to minimize the effects of the disease and to maximize the benefits of treatment.

The term “animal” as used herein includes, but is not limited to, humans and non-human vertebrates such as wild, domestic and farm animals.

The term “patient” or “subject” as used herein is an animal, particularly a human, suffering from an unwanted disease or condition that may be treated by the therapeutic and/or compositions described herein.

The term “inhibiting” generally refers to prevention of the onset of the symptoms, alleviating the symptoms, or eliminating the disease, condition or disorder.

“Optional” or “optionally” means that the subsequently described event or circumstance may or may not occur, and that the description includes instances where the event occurs and instances where it does not.

As used herein, “room temperature” means an indoor temperature of from about 20° C. to about 25° C. (68 to 77° F.).

Throughout the specification of the application, various terms are used such as “primary,” “secondary,” “first,” “second,” and the like. These terms are words of convenience in order to distinguish between different elements, and such terms are not intended to be limiting as to how the different elements may be utilized.

By “pharmaceutically acceptable,” “physiologically tolerable,” and grammatical variations thereof, as they refer to compositions, carriers, diluents, and reagents or other ingredients of the formulation, can be used interchangeably and represent that the materials are capable of being administered without the production of undesirable physiological effects such as rash, burning, irritation or other deleterious effects to such a degree as to be intolerable to the recipient thereof.

Patients with Glycogen Storage Disease type I (GSD I) may present in the neonatal period with hypoglycemia and lactic acidosis; however, they more commonly present at 3-4 months of age with hepatomegaly and/or hypoglycemic seizures. These children often have doll-like faces with excess adipose tissue in cheeks, relatively thin extremities, short stature, and a protuberant abdomen that is due to massive hepatomegaly. The hallmarks of the disease are hypoglycemia, lactic acidosis, neutropenia, hyperuricemia, and hyperlipidemia. Hypoglycemia and lactic acidemia can occur after a short fast. The histology of the liver is characterized by a universal distension of hepatocytes by glycogen and fat. The lipid vacuoles are particularly large and prominent. There is little associated fibrosis. Hepatic adenomas are known to develop in most patients with type I glycogen storage disease by the time they reach their second or third decade of life. Severe renal injury with proteinuria, hypertension, and decreased creatinine clearance due to focal segmental glomerulosclerosis and interstitial fibrosis, ultimately leading to endstage renal disease, may also be seen in young adults. GSD I has three clinical subtypes (GSD Ia, GSD Ib, and GSD Ic).

Glycogen storage disease type III (GSD III) is caused by mutations in the glycogen debranching enzyme (GDE) gene, resulting in accumulation of glycogen with short outer chains in the cytoplasm of liver and muscle cells. GSD IV, another cytoplasmic GSD caused by deficiency of glycogen branching enzyme (GBE), is characterized by the deposits of less-branched amylopectin-like polysaccharide in muscle, liver, and the central nervous system (CNS). Although both diseases have cytoplasmic glycogen accumulation, GSD III glycogen has short outer chains and is soluble, while GSD IV glycogen is less-branched. Currently there is no treatment for these diseases.

GSD III has several subtypes. Most patients have disease involving both liver and muscle (type IIIa), some (˜15% of all those with GSD-III) have only liver involvement (type IIIb), and in rare cases, there is a selective loss of only one of the two GDE activities: glucosidase (type IIIc) or transferase (type IIId). GSD IIIc affects only the muscle, and GSD IIId affects the muscle and the liver. During infancy and childhood, the dominant features are hepatomegaly, hypoglycemia, hyperlipidemia, and growth retardation. In individuals with muscle involvement (GSD IIIa), there is variable myopathy and cardiomyopathy.

GSD IV patients usually present with hepatosplenomegaly and failure to thrive in the first 18 months of life. They develop liver cirrhosis that progresses to cause portal hypertension, ascites, esophageal varices, and liver failure that leads to death by age 5 years. Some patients can develop hepatic adenomas and hepatocellular carcinoma. Carbohydrate tolerance tests and blood glucose response to glucagon or epinephrine are normal in most patients, but fasting hypoglycemia, typically present in type I and type III disease (and in some cases of type VI and type IX disease) has been observed only occasionally in this disease when liver cirrhosis progresses and few hepatocytes are available for glucose mobilization. In addition to the hepatic presentation, there is a neuromuscular presentation of type IV disease that is heterogeneous. In the childhood form, patients present predominantly with a myopathy or cardiomyopathy. The adult form can present as an isolated myopathy or as a multisystem disorder with central and peripheral nervous system dysfunction accompanied by accumulation of polyglucosan material in the nervous system (so-called adult polyglucosan body disease).

All phosphorylase kinase deficiencies are referred to as type IX glycogen storage disease. GSD IX has six subtypes and primarily involves the liver and/or muscle as shown in Table 1.

TABLE 1
GSD IX SUBTYPES

| | | | | Mutant |
|---|---|---|---|---|
| Subtype | Species | Affected Tissues | Inheritance | Gene/Subunit |
| IXa-1 | Human | Liver, blood cells | X | PHKA2/α_{L} |
| | | | chromosomal | |
| IXa-2 | Human | Liver (in blood cells, | X | PHKA2/α_{L} |
| | | normal or high) | chromosomal | |
| IXb | Human | Liver, blood cells, | autosomal | PHKB/β |
| | | muscle | | |
| IXc | Human | Liver, blood cells | autosomal | PHKG2/γ_{TL} |
| IXd | Human | Muscle | X | PHKA1/α_{M} |
| | | | chromosomal | |
| IXe | Human | Muscle | autosomal | ? |
| IXf | Human | Heart | autosomal? | ? |
| | I-Mouse* | Muscle | X | PHKA1/α_{M} |
| | | | chromosomal | |
| | gsd-Rat^{†} | Liver | autosomal | PHKG2/γ_{TL} |

GSD XI may involve the liver and/or kidney. PRKAG2 deficiency primarily manifests in the heart and skeletal muscles.

In mammalian cells there are two spatially distinct pools of glycogen: cytoplasmic and lysosomal. Glycogenolysis is the major pathway of glycogen degradation which requires two enzymes, glycogen phosphorylase and glycogen debranching enzyme, for complete degradation of cytoplasmic glycogen. A minor pathway of glycogen degradation in the lysosomes by the enzyme acid alpha-glucosidase (GAA) also plays an important role in cellular glycogen metabolism.

GSD III patients have normal GAA activity in muscle, but excessive amounts of glycogen was found not only in the cytoplasm but also in the lysosomes. Similarly, both non-membrane-bound (cytoplasmic) glycogen and membrane-bound (lysosomal-like) glycogen were found in patients with GSD IV. These observations suggest an enhanced lysosomal glycogen trafficking in GSD III/GSD IV, and the endogenous GAA activity may not be sufficient to deplete the glycogen load in the lysosomes. Administration of rhGAA may enhance glycogen clearance in lysosomes and alter the glycogen flux in the cell, thereby reducing cytoplasmic glycogen levels in GSD III/GSD IV patients.

The low abundance of the M6PR has limited rhGAA uptake in skeletal muscle of GAA-KO mice. Adjunctive therapy with β2 agonists, such as clenbuterol, can improve the efficacy of rhGAA-based ERT and gene therapy in these mice by enhancing M6PR expression in skeletal muscle and the brain. Accordingly, an adjunctive therapy with clenbuterol, a selective β2 agonists, may increase M6PR expression, enhance rhGAA uptake, and improve treatment efficacy in GSD III and GSD IV mice. This result also has clinical applications for patients with GSD III and GSD IV.

The present disclosure is directed to the administration of a lysosomal enzyme, such as GAA to reduce lysosomal glycogen in patients having a cytoplasmic glycogen storage disease, and ultimately also reduce cytoplasmic glycogen. Some of the administered GAA may go directly into the cytosol and reduce glycogen. Moreover, the development of high sustained antibody titers to rhGAA in most patients with Pompe disease has negatively impacted the therapeutic outcome including decreased efficacy and life threatening allergic responses. Such an outcome is unlikely to happen to the patients with GSD III and GSD IV because these patients express normal levels of GAA. Accordingly, the present disclosure is directed to method of treating cytoplasmic glycogen storage diseases comprising administering a lysosomal enzyme, a functional equivalent thereof, or gene therapy therewith. In some embodiments, the lysosomal enzyme may be administered in conjunction with another therapeutic or an agent that increases the efficacy or delivery of the lysosomal enzyme. In some embodiments, the lysosomal enzyme may be administered with a β2 agonist. In some embodiments, the lysosomal enzyme may be administered with an immune modulator. In some embodiments, the lysosomal enzyme may be administered with an agent to prevent hypoglycemia (e.g. cornstarch).

There are a number of enzymes involved in the synthesis and breakdown of glycogen within the body. Deficiency or dysfunction of one of these enzymes results in a group of diseases called glycogen storage diseases (GSDs), in which the clinical hallmark is excessive glycogen accumulation in various tissues. One such GSD is PRKAG2 cardiomyopathy, which is caused by mutations in the PRKAG2 gene that encodes the γ2 subunit of AMP-activated protein kinase (AMPK). AMPK is a crucial cellular energy sensor that regulates a number of vital cellular metabolic cascades and lipid/glucose metabolic pathways.

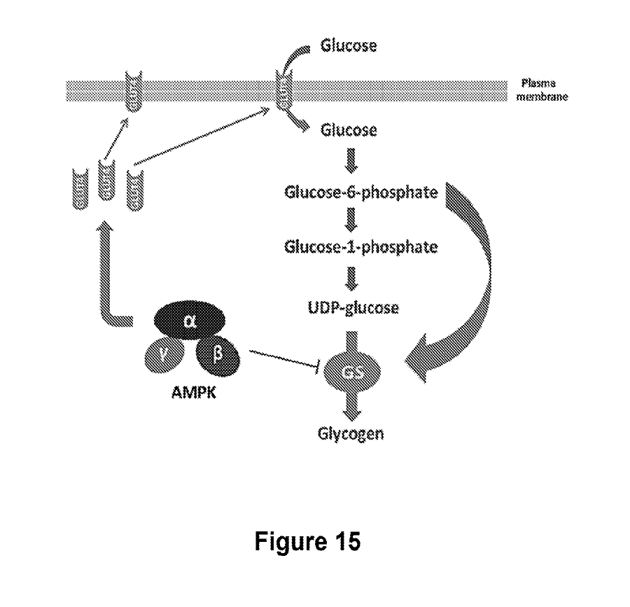

PRKAG2 cardiomyopathy is an autosomal dominant disorder with a wide spectrum of disease. The syndrome is characterized by severe infantile hypertrophic cardiomyopathy and heart rhythm disturbances at one end to cases with later presentation (age range 8 to 42 years of age) and cardiac manifestations such as increased left ventricular wall thickness and ventricular preexcitation. Other features of the disease include glycogen accumulation in skeletal muscle and the clinical spectrum of muscle involvement is being better understood with time. The underlying mechanism of excess glycogen accumulation in PRKAG2 cardiomyopathy is illustrated in FIG. 15.

As shown in FIG. 15, mutations in the PRKAG2 gene, which encodes the regulatory γ2 subunit, cause chronic activation of AMPK. Elevated AMPK activity promotes glucose transporter 4 (GLUT4) shuttling to the plasma membrane and thus induces glucose uptake and increases intracellular glucose 6-phosphate (G6P) concentration. This leads to an allosteric activation of glycogen synthase (GS), which overrides the inhibitory effect of AMPK on GS, resulting in a net increase in GS activity and excess cytoplasmic glycogen storage in cardiac muscle cells.

The clinical features of PRKAG2 cardiomyopathy closely resemble the cardiac manifestations of Pompe Disease (GSD Type II). Pompe disease is an autosomal recessive metabolic disorder that is characterized by the glycogen accumulation in lysosomes of cardiac, skeletal, and smooth muscles due to the deficiency of the lysosomal enzyme acid alpha-glucosidase (GAA). With the phenotypic similarity of PRKAG2 cardiomyopathy to Pompe disease, there is the potential for a misdiagnosis for either of these disorders, especially the infantile form of Pompe disease. Due to similar symptomatic phenotypes, rare PRKAG2 cases can be misdiagnosed with infantile Pompe disease. PRKAG2 should be considered in the differential diagnosis of cases with cardiomyopathy.

In the past, the diagnosis of Pompe disease was confirmed using GAA enzyme measurements in cultured fibroblasts or muscle cells. Enzyme measurement using acarbose, an inhibitor of alpha-glucosidase, can greatly improve the sensitivity and specificity of Pompe disease diagnosis in blood and has now been adapted in many labs as a rapid way to diagnose Pompe disease. However, without the addition of acarbose, there may be false positive results and thus, it needs to be done in labs with experience and expertise. It is believed that the diagnostic measures should be broadened to include additional tests outside of enzyme testing in dried blood spots (DBS), such as gene sequencing and measurement of GAA activity in other tissues such as skin and muscle prior to initiation of ERT.

While the present disclosure is described in detail with reference to GSD-III or GSD-IV, the methods described herein may also be used to treat individuals suffering from other GSDs, including, but not limited to glycogen storage disease type I (e.g. GSD I), glycogen storage disease III (GSD III), glycogen storage disease IV (GSD IV), glycogen storage disease V (GSD V), glycogen storage disease VI (GSD VI), glycogen storage disease VII (GSD VII), glycogen storage disease IX (GSD IX), glycogen storage disease XI (GSD XI), glycogen storage disease XII (GSD XII), glycogen storage disease XIII (GSD XIII), glycogen storage disease XIV (GSD XIV) (phosphoglucomutase deficiency), Danon disease (GSD 2B, LAMP-2 deficiency), Lafora disease, or conditions associated with protein kinase gamma subunit 2(PRKAG2)-deficiency. In some embodiments, GSD III may be selected from GSD type IIIa, type IIIb, type IIIc, or type IIId.

In addition to GSD III and GSD IV, there are no effective treatments for other cytoplasmic GSDs, including GSD I (von Gierke's disease, glucose-6-phosphatase deficiency, Ib translocase deficiency), GSD V (IIIcArdle's disease, a deficiency in muscle phosphorylase), GSD VI (Her's disease, a deficiency in liver phosphorylase), GSD VII (a deficiency in muscle phosphofructokinase; Tarui's disease), GSD IX (phosphorylase kinase deficiency), GSD XI (Franconi-Bickel syndrome; a deficiency in glucose transporter GLUT2), GSD XII (red cell aldolase deficiency; a deficiency in Aldolase A), GSD Xiii (a deficiency in b-enolase); GSD 0 (A deficiency in glycogen synthase), Lafora disease (laforin/malin deficiency), cardiac/muscle glycogenosis due to AMP-activated protein kinase gamma subunit 2-deficiency (PRKAG2 cardiac syndrome), GSD XIV due to phosphoglucomutase deficiency; and Danon disease (GSD 2B) due to LAMP-2 deficiency.

Accordingly, some embodiments of the present disclosure provide for a method of treating a cytoplasmic glycogen storage disorder comprising administering a lysosomal enzyme to an individual in need thereof. In some embodiments, the cytoplasmic glycogen storage disorder may be selected from glycogen storage disease type I (GSD I), glycogen storage disease III (GSD III), glycogen storage disease IV (GSD IV), glycogen storage disease V (GSD V), glycogen storage disease VI (GSD VI), glycogen storage disease VII (GSD VII), glycogen storage disease IX (GSD IX), glycogen storage disease XI (GSD XI), glycogen storage disease XII (GSD XII), glycogen storage disease XIII (GSD XIII), glycogen storage disease XIV (GSD XIV) (phosphoglucomutase deficiency), Danon disease (GSD 2B, LAMP-2 deficiency), Lafora disease, conditions associated with PRKAG2 deficiency, any other condition where there is cytoplasmic accumulation of glycogen, or a combination thereof.

In some embodiments, the subject to be treated has a primarily hepatic form of the cytoplasmic glycogen storage disease to be treated. In some embodiments, the subject primarily has mainly hepatic and/or cardiac involvement of the cytoplasmic glycogen storage disease to be treated. In some embodiments, the cytoplasmic glycogen storage disease is in its early stages. In some embodiments, the subject does not have a significant amount of fibrosis.

Some embodiments herein are directed to the use of a lysosomal enzyme for the treatment of conditions associated with PRKAG2 deficiency. In some embodiments, a method of treating a condition associated with PRKAG2 deficiency in an individual comprises administering to the individual a therapeutically effective amount of a lysosomal enzyme. In some embodiments, the condition is selected from hypotonia, cardiomyopathy, myopathy, cytoplasmic glycogen accumulation, ventricular hypertrophy, severe infantile hypertrophic cardiomyopathy, heart rhythm disturbances, increased left ventricular wall thickness, ventricular preexcitation, or a combination thereof.

Some embodiments are directed to a method of improving motor skills in an individual with a PRKAG2 gene mutation comprising administering to the individual a therapeutically effective amount of acid alpha-glucosidase. Some embodiments are directed to a method of improving muscle strength and function in an individual with a PRKAG2 gene mutation comprising administering to the individual a therapeutically effective amount of acid alpha-glucosidase. Some embodiments are directed to a method of decreasing seizures in an individual with a PRKAG2 gene mutation comprising administering to the individual a therapeutically effective amount of acid alpha-glucosidase.

Some embodiments are directed to a method of treating cardiac hypertrophy in an individual with a PRKAG2 gene mutation comprising administering to the individual a therapeutically effective amount of acid alpha-glucosidase. Some embodiments are directed to a method of treating cardiomyopathy in an individual with a PRKAG2 gene mutation comprising administering to the individual a therapeutically effective amount of acid alpha-glucosidase. Some embodiments are directed to a method of treating myopathy in an individual with a PRKAG2 gene mutation comprising administering to the individual a therapeutically effective amount of acid alpha-glucosidase.

In some embodiments, a therapeutic agent may be administered in combination with (e.g. prior to, after, and/or concurrently with) the lysosomal enzyme. In some embodiments, the therapeutic agent may be selected from a growth hormone, an autocrine glycoprotein, a β2 agonist, an agent to treat or prevent hypoglycemia (e.g. cornstarch), an agent to treat or prevent neutropenia, an agent to suppress glycogen synthase (e.g. RNAi; 20(S)-protopanaxadiol), an agent to prevent or reverse glycogen synthesis, an agent to treat or prevent fibrosis, an agent to improve mitochondrial function, an agent to treat any other symptom of the cytoplasmic storage disorders of embodiments herein, or a combination thereof.

In some embodiments, the β2 agonist is a selective β2 agonist. In some embodiments, the β2 agonist is albuterol, arbutamine, bambuterol, befunolol, bitolterol, bromoacetylalprenololmenthane, broxaterol, carbuterol, cimaterol, cirazoline, clenbuterol, clorprenaline, denopamine, dioxethedrine, dopexamine, ephedrine, epinephrine, etafedrine, ethylnorepinephrine, etilefrine, fenoterol, formoterol, hexoprenaline, higenamine, ibopamine, isoetharine, isoproterenol, isoxsuprine, mabuterol, metaproterenol, methoxyphenamine, norepinephrine, nylidrin, oxyfedrine, pirbuterol, prenalterol, procaterol, propranolol, protokylol, quinterenol, ractopamine, reproterol, rimiterol, ritodrine, salmefamol, soterenol, salmeterol, terbutaline, tretoquinol, tulobuterol, xamoterol, zilpaterol, zinterol, or a combination thereof. In some embodiments, the β2 agonist may be clenbuterol. In some embodiments, the β2 agonist is clenbuterol, albuterol, formoterol, salmeterol, or a combination thereof. The β2 agonist may be administered bimonthly, monthly, biweekly, weekly, twice weekly, daily, twice a day, three times a day, or more often a day. In some embodiments, the β2 agonist is administered in an amount of about 20 μg per day to about 2100 μg per day.

In some embodiments, the acid alpha-glucosidase and the β2 agonist are components of separate pharmaceutical compositions that are administered separately. In some embodiments, the β2 agonist and the acid alpha-glucosidase are components of separate pharmaceutical compositions that are mixed together before administration. In some embodiments, the β2 agonist is administered separately prior to, concurrently with, or subsequent to administration of the acid alpha-glucosidase. In some embodiments, the β2 agonist and the acid alpha-glucosidase are in a single pharmaceutical composition.

Some embodiments provide for a method of treating a cytoplasmic glycogen storage disorder comprising administering an adjunctive therapy comprising a therapeutic agent of embodiments herein to enhance efficacy of a lysosomal enzyme.

In some embodiments, the lysosomal enzyme is selected fromglucocerebrosidase, acid alpha-glucosidase, alpha-galactosidase, alpha-n-acetylgalactosaminidase, acid sphingomyelinase, alpha-iduronidase, or a combination thereof. In some embodiments, the lysosomal enzyme may be acid alpha-glucosidase. The acid α-glucosidase may be selected from GAA, alglucosidase alfa, recombinant human acid alpha-glucosidase (rhGAA), neo-rhGAA, reveglucosidase alpha, an rhGAA administered with a chaperone (e.g. 1-deoxynojirimycin (DNJ), α-homonojirimycin, or castanospermine), or a combination thereof. In some embodiments, the acid alpha-glucosidase is administered bimonthly, monthly, biweekly, weekly, twice weekly, daily, twice a day, three times a day, or more often a day. In some embodiments, the acid alpha-glucosidase is administered in a therapeutically effective amount. In some embodiments, the therapeutically effective amount is about 1 mg/kg to about 50 mg per kg bodyweight of the individual. In some embodiments, the lysosomal enzyme may be administered in a higher dose initially to clear the glycogen load before administering the lysosomal enzyme.

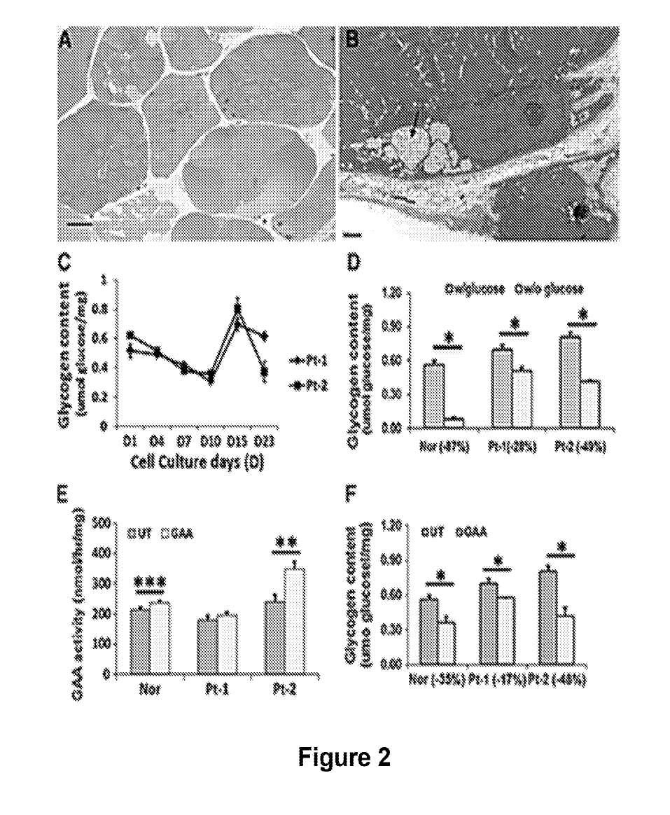

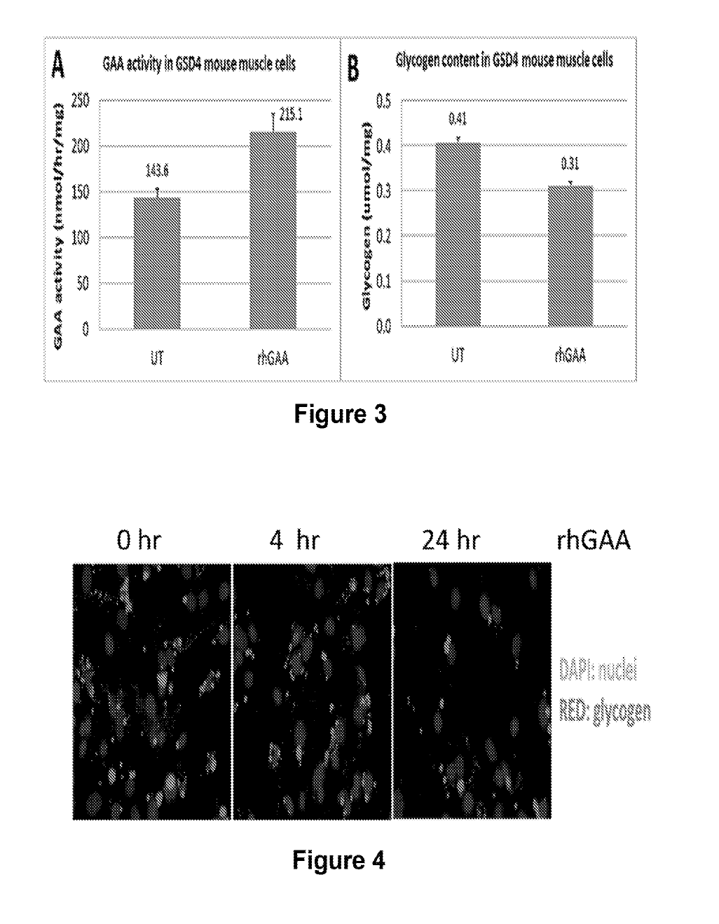

Results have shown that recombinant human acid alpha-glucosidase (rhGAA) significantly reduced glycogen content in primary muscle cells from GSD IIIc patients (FIG. 2) and in the primary myoblasts from GSD IV mice (FIGS. 3 & 4) in vitro It is believed that enhanced GAA activity leads to rapid lysosomal glycogen clearance, increased glycogen shuffling from cytoplasm into lysosomes, and a reduced overall cytoplasmic glycogen level in the affected tissues of GSD III and IV.

In some embodiments, the lysosomal enzyme may be administered to the individual in a form that, when administered, targets tissues such as the tissues affected by the disease (e.g., liver, heart or muscle). In some embodiments, the lysosomal enzyme is administered in its precursor form. In some embodiments, a mature form of the lysosomal enzyme (e.g. GAA) that has been modified to contain motifs to allow efficient uptake of the lysosomal enzyme may be administered.

In embodiments, the lysosomal enzyme may be selected from glucocerebrosidase (for the treatment of Gaucher disease; U.S. Pat. No. 5,879,680 and U.S. Pat. No. 5,236,838,) alpha-glucosidase (e.g., acid alpha-glucosidase) (for the treatment of Pompe disease; PCT International Publication No. WO 00/12740), alpha-galactosidase (e.g., alpha-gal, alpha-galactosidase or alpha-gal) (for the treatment of Fabry Disease; U.S. Pat. No. 5,401,650), alpha-n-acetylgalactosaminidase (for the treatment of Schindler Disease; U.S. Pat. No. 5,382,524), acid sphingomyelinase (for the treatment of Niemann-Pick disease; U.S. Pat. No. 5,686,240), alpha-iduronidase (for the treatment of Hurler, Scheie, or Hurler-Scheie disease; PCT International Publication No. WO 93/10244A1), or a combination thereof.

In some embodiments, the lysosomal enzyme is acid alpha-glucosidase (GAA). In some embodiments, the GAA may be human. In some embodiments, the human GAA is administered in its precursor form, as the precursor contains motifs which allow efficient receptor-mediated uptake of GAA. Alternatively, a mature form of human GAA that has been modified to contain motifs to allow efficient uptake of GAA, can be administered. In some embodiments, the GAA is recombinant GAA. In some embodiments, the GAA is a precursor form of recombinant human GAA (rhGAA). In some embodiments, the GAA is GAA, rhGAA, alglucosidase alfa, neo-rhGAA (modified recombinant human GAA with synthetic oligosaccharide ligands which is sold by Genzyme Corp.), reveglucosidase alpha (a fusion of IGF-2 and GAA sold by Biomarin Pharmaceuticals, Inc.), ATB200 (an rhGAA with a higher bis-M6P content) that is administered in combination with AT221 (an oral chaperone molecule—(e.g. 1-deoxynojirimycin (DNJ), α-homonojirimycin, or castanospermine)) (sold by Amicus Therapeutics, Inc.), a portion thereof, or a combination thereof. The rhGAA may be alglucosidase alfa (sold by Genzyme Corp. under the tradename Myozyme® (for infantile onset Pompe disease) and Lumizyme®).

GAA may be obtainable from a variety of sources. In some embodiments, a recombinant human acid α-glucosidase (rhGAA) produced in Chinese hamster ovary (CHO) cell cultures is used (see, e.g., Fuller, M. et al., Eur. J. Biochem. 234:903 909 (1995); Van Hove, J. L. K. et al., Proc. Natl. Acad. Sci. USA 93:65 70 (1996) and U.S. Pat. No. 7,056,712). Production of GAA in CHO cells yields a product having glycosylation that allows significant and efficient uptake of GAA in tissues such as heart and muscle. In some embodiments, Myozyme® ((alglucosidase alfa) Genzyme Corp.), or other recombinant human GAA, may be used in accordance with the embodiments described herein.

In embodiments, the GAA may have a specific enzyme activity in the range of about 1.0 to about 8.0 μmol/min/mg protein, about 2.0 to about 8.0 μmol/min/mg protein, about 3.0-8.0 μmol/min/mg protein, about 4.0 to about 8.0 μmol/min/mg protein, about 2.0 to about 3.5 μmol/min/mg protein, about 1.0 to about 3.5 μmol/min/mg protein, about 1.0 to about 5 μmol/min/mg protein, about 2.0 to about 5 μmol/min/mg protein, or a range between any two of these values. In some embodiments, the GAA has a specific enzyme activity of at least about 1.0 μmol/min/mg protein, at least about 2.0 μmol/min/mg protein, at least about 2.5 μmol/min/mg protein, at least about 2.75 μmol/min/mg protein, at least about 3.0 μmol/min/mg protein, at least about 3.5 μmol/min/mg protein, at least about 4.0 μmol/min/mg protein, at least about 5.0 μmol/min/mg protein, at least about 6.0 μmol/min/mg protein, at least about 7.0 μmol/min/mg protein, at least about 8.0 μmol/min/mg protein, or a range between any two of these values.

According to some embodiments, a method of treating a cytoplasmic glycogen storage disorder may include increasing expression of receptors for the lysosomal enzyme, or otherwise increasing cell surface density of such receptors, in an individual in need thereof. Accordingly, in some embodiments, a method of treating a cytoplasmic glycogen storage disorder of embodiments herein comprises administering an adjunctive therapy comprising a therapeutic agent to enhance the efficacy of a lysosomal enzyme. In some embodiments, a method of treating a cytoplasmic glycogen storage disorder of embodiments herein comprises administering a lysosomal enzyme and another therapeutic agent. In some embodiments, a method of treating a cytoplasmic glycogen storage disorder of embodiments herein comprises administering a therapeutic agent as an adjunctive therapy to lysosomal enzyme replacement therapy. In some embodiments, the therapeutic agent may be selected from a growth hormone, an autocrine glycoprotein, a β2 agonist, an agent to treat or prevent hypoglycemia (e.g. cornstarch), an agent to treat or prevent neutropenia, an agent to suppress glycogen synthase (e.g. RNAi; 20(S)-protopanaxadiol), an agent to prevent or reverse glycogen synthesis, an agent to treat or prevent fibrosis (e.g. PDE4 inhibitors), an agent to improve mitochondrial function, an agent to treat any other symptom of the cytoplasmic storage disorders of embodiments herein, or a combination thereof. Therapeutic agents of embodiments herein may selectively modulate expression of receptors for particular lysosomal enzymes. Expression of receptors for a lysosomal enzyme may also be increased by behaviors, such as exercise. In some embodiments, a β2 agonist may be administered to an individual suffering from adult-onset or late-onset glycogen storage disease II, or a patient who presents with only partial enzyme deficiency, wherein administering the β2 agonist results in biochemical correction of the enzyme deficiency in target tissues and improved motor function.

In some embodiments, the lysosomal enzyme may be administered alone, or in compositions or medicaments comprising the lysosomal enzyme, as described herein. In some embodiments, for the treatment of cytoplasmic glycogen storage disorders, a therapeutic agent of embodiments described herein may be administered to a patient in combination with a lysosomal enzyme. In some embodiments, a therapeutic agent and lysosomal enzyme may be components of a single pharmaceutical composition. In some embodiments, a therapeutic agent and lysosomal enzyme may be components of separate pharmaceutical compositions that are mixed together before administration. In some embodiments, the therapeutic agent and lysosomal enzyme may be components of separate pharmaceutical compositions that are administered separately. In some embodiments, the therapeutic agent and the lysosomal enzyme may be administered simultaneously, without mixing (e.g., by delivery of the β2 agonist on an intravenous line by which the lysosomal enzyme is also administered). In some embodiments, the therapeutic agent may be administered separately (e.g., not admixed), but within a short time frame (e.g., within 24 hours) prior to or subsequent to administration of the lysosomal enzyme. A synergistic effect may support reduced dosing of ERT when used with the therapeutic agent and a reduced dosing of the therapeutic agent.

In embodiments, a lysosomal enzyme, such as GAA, may be administered in a form that targets tissues such as the tissues affected by the disease (e.g., heart, muscle, brain). The lysosomal enzyme may be optionally administered in conjunction with other agents, such as antihistamines or immunosuppressants or other immunotherapeutic agents, such as methotrexate, that counteract anti-lysosomal enzyme antibodies. In embodiments, the lysosomal enzymes may include a human enzyme, recombinant enzyme, wild-type enzyme, synthetic enzyme, or a combination thereof.

In the embodiments described herein, a therapeutically effective amount of the lysosomal enzyme is administered. In some embodiments, the lysosomal enzyme is administered as part of a lysosomal enzyme replacement therapy. In some embodiments, the therapeutically effective amount of the lysosomal enzyme (e.g. GAA) is about 1 mg/kg to about 100 mg/kg, about 1 mg/kg to about 75 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 5 mg/kg to about 100 mg/kg, about 5 mg/kg to about 75 mg/kg, about 5 mg/kg to about 60 mg/kg, about 5 mg/kg to about 50 mg/kg, about 5 mg/kg to about 40 mg/kg, about 5 mg/kg to about 30 mg/kg, about 5 mg/kg to about 20 mg/kg, about 10 mg/kg to about 100 mg/kg, about 10 mg/kg to about 75 mg/kg, about 10 mg/kg to about 60 mg/kg, about 10 mg/kg to about 50 mg/kg, about 10 mg/kg to about 40 mg/kg, about 10 mg/kg to about 30 mg/kg, about 10 mg/kg to about 20 mg/kg, less than about 100 mg/kg, less than about 75 mg/kg, less than about 60 mg/kg, less than about 50 mg/kg, less than about 40 mg/kg, less than about 30 mg/kg, less than about 25 mg/kg, less than about 20 mg/kg, less than about 15 mg/kg, less than about 10 mg/kg, less than about 5 mg/kg, or a range between any two of these values. In some embodiments, the effective dosage may be about 20 mg/kg, about 25mg/kg, about 30 mg/kg, about 40 mg/kg, about 45 mg/kg, about 50 mg/kg, about 60 mg/kg, about 70 mg/kg, about 80 mg/kg, about 90 mg/kg, about 100 mg/kg, or a range between any two of these values. In some embodiments, the effective dose for a particular individual may be varied (e.g., increased or decreased) over time, depending on the needs of the individual. For example, in times of physical illness or stress, or if anti-enzyme antibodies become present or increase, or if disease symptoms worsen, the amount may be increased. As another example, an increased effective dose may be administered (perhaps weekly) initially to clear the glycogen load before administering a reduced effective dosage. In some embodiments, the type of lysosomal enzyme delivered may be varied over time, depending on the needs of the individual. For example, initially, a more potent form of GAA may be administered (e.g. neo-GAA or reveglucosidase) followed by administration of a less potent but perhaps more cost-effective GAA type (e.g. rhGAA).

In embodiments, the therapeutically effective amount of the lysosomal enzyme (or composition or medicament containing the lysosomal enzyme) may be administered at regular intervals, depending on the nature and extent of the disease's effects, and on an ongoing basis. Administration at a “regular interval,” as used herein, indicates that a therapeutically effective amount is administered periodically (as distinguished from a one-time dose). The interval can be determined by standard clinical techniques. In some embodiments, the lysosomal enzyme's periodic administrations may be bimonthly, monthly, biweekly, weekly, twice weekly, daily, twice a day, three times a day, or more often a day. The administration interval for a single individual need not be a fixed interval, but can be varied over time, depending on the needs of the individual. For example, in times of physical illness or stress, if anti-enzyme antibodies become present or increase, or if disease symptoms worsen, the interval between doses may be decreased. In some embodiments, a therapeutically effective amount of the lysosomal enzyme at an amount of about 40 mg/kg body weight may be administered weekly. In some embodiments, a therapeutically effective amount of the lysosomal enzyme at an amount of about 20 mg/kg body weight may be administered twice weekly. In some embodiments, a therapeutically effective amount of the lysosomal enzyme at an amount of about 45 mg/kg body weight may be administered weekly. In some embodiments, a therapeutically effective amount of the lysosomal enzyme at an amount of about 22.5 mg/kg body weight may be administered twice weekly.

In some embodiments, the lysosomal enzyme may be administered once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or a range between any two of these values. In some embodiments, the lysosomal enzyme may be administered at least once every 1, 2, 3, 4, 5, 6, 7, 8, 9, 10, 11, 12, 13, 14, 15, 16, 17, 18, 19, 20 weeks, or a range between any two of these values. In some embodiments, the lysosomal enzyme may be administered using single or divided doses of every 60, 48, 36, 24, 12, 8, 6, 4, or 2 hours, or a range between any two of these values, or a combination thereof. For example, in some embodiments, the lysosomal enzyme, functional equivalent thereof, or gene may be administered once every about one to about two, about two to about three, about three to about four, or about four to about five weeks.

In some embodiments, the lysosomal enzyme (or composition or medicament containing the lysosomal enzyme) is administered by an appropriate route. The therapeutic agents of embodiments herein may be administered by any suitable route, including administration by inhalation or insufflation (either through the mouth or the nose) or oral, sublingual, buccal, parenteral, topical, subcutaneous, intraperitoneal, intraveneous, intrapleural, intraoccular, intraarterial, rectal administration, or within/on implants, e.g., matrices such as collagen fibers or protein polymers, via cell bombardment, in osmotic pumps, grafts comprising appropriately transformed cells, etc. In one embodiment, the lysosomal enzyme may be administered intravenously. In other embodiments, the lysosomal enzyme may be administered by direct administration to a target tissue, such as heart or muscle (e.g., intramuscular). In yet another embodiment, the lysosomal enzyme is administered orally. More than one route can be used concurrently, if desired.

In some embodiments, administration of a lysosomal enzyme may also encompass administration of a functional equivalent of a lysosomal enzyme. A functional equivalent may include a compound different from the lysosomal enzyme that, when administered to the patient, replaces the function of the lysosomal enzyme to treat the cytoplasmic glycogen storage disorder. Such functional equivalents may include mutants, analogs, and derivatives of lysosomal enzymes.

β2 agonists are molecules that stimulate the β2-adrenergic receptor. Numerous β2 agonists are known in the art and may be used in the therapeutic methods of the embodiments described herein. In some embodiments, the β2 agonist used in embodiments herein may be selected from albuterol, arbutamine, bambuterol, befunolol, bitolterol, bromoacetylalprenololmenthane, broxaterol, carbuterol, cimaterol, cirazoline, clenbuterol, clorprenaline, denopamine, dioxethedrine, dopexamine, ephedrine, epinephrine, etafedrine, ethylnorepinephrine, etilefrine, fenoterol, formoterol, hexoprenaline, higenamine, ibopamine, isoetharine, isoproterenol, isoxsuprine, mabuterol, metaproterenol, methoxyphenamine, norepinephrine, nylidrin, oxyfedrine, pirbuterol, prenalterol, procaterol, propranolol, protokylol, quinterenol, ractopamine, reproterol, rimiterol, ritodrine, salmefamol, soterenol, salmeterol, terbutaline, tretoquinol, tulobuterol, xamoterol, zilpaterol, zinterol, or a combination thereof. In some embodiments, β2 agonists used in the disclosed methods do not interact, or show substantially reduced interaction, with β1-adrenergic receptors. In some embodiments, the β2 agonist is a selective β2 agonist. In embodiments, the β2 agonist is clenbuterol, albuterol, formoterol, salmeterol, or a combination thereof. In embodiments, the β2 agonist is clenbuterol. In embodiments, the β2 agonist is albuterol.

In some embodiments, the therapeutic agent (e.g. β2 agonist) may be administered at a dosage of, for example, 0.1 to 100 mg/kg, such as 0.5, 1.0, 1.1, 1.6, 2, 4, 8, 9, 10, 11, 15, 16, 17, 18, 19, 20, 21, 22, 27, 28, 29, 30, 40, 45, 50, 60, 70, 80, 90 or 100 mg/kg per day, or a range between any two of these values. Dosage forms suitable for internal administration may contain from about 0.1-500 milligrams of active ingredient per unit. In these pharmaceutical compositions, in some embodiments, the active ingredient may be present in an amount of about 0.5-95% by weight based on the total weight of the composition.

In the embodiments described herein, a therapeutically effective amount of clenbuterol may be administered. In some embodiments, the therapeutically effective amount of clenbuterol is about 80 μg/day to about 160 μg/day. In some embodiments, the therapeutically effective amount of clenbuterol is about 20 μgg/day to about 2100 μg/day, about 20 μg/day to about 720 μg/day, about 20 μg/day to about 500 μg/day, about 20 μg/day to about 300 μg/day, about 20 μg/day to about 200 μg/day, about 40 μg/day to about 2100 μg/day, about 40 μg/day to about 720 μg/day, about 40 μg/day to about 500 μg/day, about 40 μg/day to about 300 μg/day, about 40 μg/day to about 200 μg/day, about 80 μg/day to about 2100 μg/day, about 80 μg/day to about 720 μg/day, about 80 μg/day to about 500 μg/day, about 80 μg/day to about 300 μg/day, about 80 μg/day to about 200 μg/day, or a range between any two of these values. In embodiments, the effective amount for a particular individual may be varied (e.g., increased or decreased) over time, depending on the needs of the individual.

In some embodiments, a therapeutic agent may be administered once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or a range between any two of these values. In some embodiments, a therapeutic agent may be administered at least once every 1, 2, 3, 4, 5, 6, 7, 8, 9, 10, 11, 12, 13, 14, 15, 16, 17, 18, 19, 20 weeks, or a range between any two of these values. In some embodiments, the therapeutic agent may be administered using single or divided doses of every 60, 48, 36, 24, 12, 8, 6, 4, or 2 hours, or a range between any two of these values, or a combination thereof.

Table 2 shows exemplary therapeutic agents, dosage, route of administration and frequency. This table is exemplary, and is not meant to be limiting.

TABLE 2
Exemplary therapeutic agents

| Name | Dose | Route | Frequency |
|---|---|---|---|
| Bambuterol | Adult: 10 mg, increased to 20 mg | Oral solution | daily for children and |
| | after 1-2 wk. | | adults |
| | Child (6-12 yr.): 5 mg, may be | | |
| | increased after 1-2 wk. (>10 not | | |
| | recom. In oriental children) | | |
| | Child (2-5 yr.): 5 mg | | |
| Bitolterol | Intermittent Nebulization: 0.5- | Inhalation | 2 inhalations every 8 hr. |
| (Adult and Child the | 1.5 mg, severe patients (2 mg- | | Do not exceed 3 |
| same) | 8 mg). | | inhalations every 6 hr. or |
| | Continuous Nebulization: 2.5 | | 2 inhalations every 4 hr. |
| | mg w/max. of 14 mg | | |
| Ephedrine (Adults | Oral/Subcutaneous: (Initial | Oral, | As needed; 150 mg/day |
| only) | Dose) 25-50 mg | Subcutaneous, | (max dose) |
| | IV: 5-25 mg (over 15 min.) | IV | |
| Ephedrine (Child | 2-3 mg/kg | Oral, | daily, divided up into 4-6 |
| only >2 yr.) | | Subcutaneous | doses |
| Epinephrine (Adult | 0.3 mg | IV, Inhalation, | As needed |
| only) | 0.5 mL or one vial (nebulizer) | Intaspinal | |
| | (0.1 mg/mL solution) 0.1 to 1 | | |
| | mg | | |
| Epinephrine (Child | 0.15 mg | IV, Inhalation | As needed, 0.5 mg/dose |
| only) | (1.0 mg/mL) 0.01 mg/kg | | (max dose) |
| | (0.1 mg/mL) 0.005-0.01 mg/kg | | |
| Ethylnorepinephrine | 0.5-1 mL | Injection | As recommended |
| Etilefrine (Adult | Injection: 10 mg | Injection, IV | Injection: every 1-3 hr., if |
| only) | IV: 0.2-0.6 mg/min. | | necessary |
| Etilefrine (Child | <2 yr.- | Injection, IV | Injection: every 1-3 hr., if |
| only) | Injection: 2-4 mg | | necessary |
| | IV: 0.05-0.2 mg/min. | | |
| | 2-6 yr.- | | |
| | Injection: 4-7 mg | | |
| | IV: 0.1-0.4 mg/min. | | |
| | >6 yr.- | | |
| | Injection: 7-10 mg | | |
| | IV: 0.2-0.5 mg/min. | | |
| Fenoterol (Adult | Inhalation-0.007-0.035 mg/kg | Inhalation, Oral | Inhalation-every 6 hr. |
| only) | Oral-100-200 mcg | | Oral-every 8 hr. |
| Fenoterol (Child | Inhalation-8 μg/kg | Inhalation | every 8 hr. |
| only) | | | |
| Formoterol (Adult | 12 mcg of powder | Inhalation | 15 min. before exercise, |
| only) | 12 mcg inhalation capsule or 20 | | every 12 hr.; 24 mcg (max |
| | mcg/2 mL inhalation solution | | dose) |
| | | | every 12 hrs. for |
| | | | inhalation capsule (24 |
| | | | mcg max dose) and every |
| | | | 12 hrs. for inhalation |
| | | | solution |
| Formoterol (Child | 12 mcg of powder | Inhalation | every 12 hr., 24 mcg (max |
| only) | | | dose) |
| Isoetharine (Adult | 0.005-0.09 mg/kg | Inhalation | Every 4 hrs. |
| only) | | | |
| Isoproterenol (Adult | 1:200 solution: 5-15 deep | Inhalation, IV | If relief is not observed, |
| only) | inhalations. | | repeat dosing. Repeat up |
| | 1:100 solution: 3-7 deep | | to 5x/day. |
| | inhalations | | Initial dose may be |
| | Dilute 1 mL to 10 mL W/NaCl | | repeated when necessary |
| | Dilute 5 mL in 500 mL in 5% | | administer at 5 mcg/min. |
| | dextrose injection | | 0.5-5 mcg/min. |
| Isoproterenol (Child | 1:200 solution: 5-15 deep | Inhalation | Asthma (Acute)-If relief |
| only) | inhalations. Do not use more | | is not observed, repeat |
| | than 0.25 mL of 1:200 solution | | dosing. Repeat up to |
| | during one treatment. | | 5x/day. |
| Metaproterenol | Oral: 20 mg | Oral, Inhalation | Oral: 3-4x/day |
| (Adult only) | Inhalation aerosol: 2-3 | | Inhalation aerosol: every |
| | inhalations | | 3-4 hrs. up to 12 |
| | Inhalation solution: 10-15 mg | | inhalations/day |
| | | | Inhalation solution: every |
| | | | 3-6 hr. |
| Metaproterenol | Infant and children (Inhalation) | Oral, Inhalation | Infant and children |
| (Child only) | (<12 yr.): 0.5-1 mg/kg; min. dose: | | (Inhalation) (<12 yr.): |
| | 5 mg; max. dose: 15 mg | | every 4-6 hr. |
| | Infant and children (Oral) | | Infant and children (Oral) |
| | (<2 yr).: 0.4 mg/kg/dose | | (<2 yr).: dose divided into |
| | Children (Oral) (2-6 yr.): 1.3- | | 3-4x/day (Children); |
| | 2.6 mg/kg/day | | divided into 8-12x/day |
| | Children (Oral)(6-9 yr.): 10 mg | | (Infants) |
| | Children (oral)(>9 yr.): 20 mg | | Children (Oral) (2-6 yr.): |
| | | | divided every 6-8 hr. |
| | | | Children (Oral)(6-9 yr.): |
| | | | 3-4x/day |
| | | | Children (oral)(>9 yr.): 3- |
| | | | 4x/day |
| Norepinephrine | Initial dose: 2-4 mcg/min | IV | Initial dose: daily |
| (Adult only) | Maintenance dose: avg. 1-12 | | Maintenance dose: daily |
| | mcg/min. (based on rate for low | | |
| | normal blood pressure) | | |
| Nylidrin (Adult | 3-12 mg | Oral | 3-4x/day |
| only) | | | |
| Pirbuterol (Adult | 0.4 mg | Inhalation | repeated every 4-6 hr. |
| and child) | | | |
| Propranolol | Intial Dose: 40 mg (Immed. | Oral, IV | Intial dose: 2x/day |
| | Release); 80 mg (Sustained | | (Immed. Release); 1x/day |
| | Release) | | (Sustained Release) |
| | Maintenance Dose: 120-240 mg | | Maintenance dose: 1x/day |
| | (Immed. Release); 120-160 mg | | (Immed. And Sustained |
| | (Sustained Release) | | Release) |
| | Immed. Release: 80-320 mg | | Immed Release: Doses |
| | (total dose) | | divided into 2-4x/day |
| | Sustained Release: (avg. optimal | | Sustained Release: daily |
| | dose) 160 mg | | 3-4x/day (oral); rate not |
| | 10-30 mg (oral); 1-3 mg (IV) | | exceeding 1 mg/min (IV) |
| | Initial dose: 40 mg | | Intial Dose: 3x/day for 1 |
| | Maintenance: 180-240 mg | | month |
| | Intial Dose: 80 mg (Immed. | | Maintenance: 2-4x/day in |
| | Release); 80 mg (Sustained | | divided doses |
| | Release) | | Intial Dose: per day in |
| | Maintenance Dose: 160-240 mg | | divided doses (for immed. |
| | (immed. and Sustained Release) | | and sustained release) |
| | | | Maintenance: daily |
| Ritodrine (Adults | Capsules: 40 mg | Oral, Injection | Capsules: every 8-12 hrs. |
| only) | Tablets: 10-20 mg | | Tablets: every 4-6 hrs. |
| | Injection: 50-350 mcg/min | | |
| Salmeterol (Adults | 50 mcg | Inhalation | every 12 hr. |
| and Child) | | | |
| Terbutaline | IV: 0.08-6 mcg/kg/min | Oral, IV, | Inhalation. 60 sec. apart, |
| | Subcutaneous Inj.: 0.25 mg | Injection | every 2-6 hr. |
| | Inhalation. 2 inhalations | | Sub. Inj.: As needed every |
| | Oral: 2.5-7.5 mg | | 15-30 min., do not exceed |
| | | | 0.4 mg in 4 hr., or every 6 |
| | | | hr. |
| | | | Oral: 3x/day at 6 hr. |
| | | | Intervals, do not exceed |
| | | | 15 mg in 24 hr. |
| | | | IV: max dose 80 mcg/min. |
| Clenbuterol | 40 μg/day up to 160 μg/day; 40 | oral, tablet and | daily or twice daily |
| | μg once daily; 40 μg BID; 80 μg | syrup | |
| | in the morning, 40 μg in the | | |
| | evening; 80 μg BID | | |
| Albuterol | 4 mg/day up to 16 mg/day oral; 4 | oral, tablet and | daily or twice daily |
| | mg once daily, 4 mg twice daily, | syrup | |
| | 4 mg in the morning and 8 mg in | | |
| | the evening, 8 mg twice daily. | | |

As known by those of skill in the art, the optimal dosage of therapeutic agents useful in embodiments herein depends on the age, weight, general health, gender, and severity of the cytoplasmic glycogen storage disorder of the individual being treated, as well as route of administration and formulation. A skilled practitioner is able to determine the optimal dose for a particular individual. Additionally, in vitro or in vivo assays may be employed to help to identify optimal dosage ranges, for example, by extrapolation from dose-response curves derived from in vitro or animal model test systems.

In some embodiments, the compositions may be formulated with a physiologically acceptable carrier or excipient to prepare a pharmaceutical composition. Suitable pharmaceutically acceptable carriers may include, but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations may, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In some embodiments, a water-soluble carrier suitable for intravenous administration may be used.

In some embodiments, the lysosomal enzyme may be formulated as neutral or salt forms. Pharmaceutically acceptable salts may include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The composition or medicament, if desired, may also contain minor amounts of wetting or emulsifying agents, or pH buffering agents. In some embodiments, the composition may be a liquid solution, suspension, emulsion, tablet, pill, capsule, sustained release formulation, or powder. In some embodiments, the composition may also be formulated as a suppository, with traditional binders and carriers such as triglycerides. Oral formulation may include standard carriers such as pharmaceutical grades of mannitol, lactose, starch, magnesium stearate, polyvinyl pyrollidone, sodium saccharine, cellulose, magnesium carbonate, etc.

The composition or medicament can be formulated in accordance with the routine procedures as a pharmaceutical composition adapted for administration to human beings. For example, in some embodiments, a composition for intravenous administration may be a solution in sterile isotonic aqueous buffer. In some embodiments, the composition can also include a solubilizing agent and a local anesthetic to ease pain at the site of the injection. In some embodiments, the ingredients may be supplied either separately or mixed together in unit dosage form, for example, as a dry lyophilized powder or water free concentrate in a hermetically sealed container, such as an ampule or sachette indicating the quantity of active agent. In some embodiments, where the composition is to be administered by infusion, it may be dispensed with an infusion bottle containing sterile pharmaceutical grade water, saline or dextrose/water. In some embodiments, where the composition is administered by injection, an ampule of sterile water for injection or saline can be provided so that the ingredients may be mixed prior to administration.

For gene therapy, genes encoding the aforesaid lysosomal enzymes may be used.

The methods of the present disclosure contemplate single as well as multiple administrations, given either simultaneously or over an extended period of time. In embodiments, a therapeutic agent may be administered at regular intervals (i.e., periodically) and on an ongoing basis, depending on the nature and extent of effects of the cytoplasmic glycogen storage disorder, and also depending on the outcomes of the treatment. In some embodiments, a therapeutic agent's periodic administrations may be bimonthly, monthly, biweekly, weekly, twice weekly, daily, twice a day, three times a day, or more often a day. Administrative intervals may also be varied, depending on the needs of the patient. For example, in some embodiments, in times of physical illness or stress, if anti-lysosomal enzyme antibodies become present or increase, or if disease symptoms worsen, the interval between doses may be decreased. Therapeutic regimens may also take into account half-life of the administered therapeutic agents of embodiments herein.

In some embodiments, a therapeutic agent may be administered prior to, or concurrently with, or shortly thereafter, the lysosomal enzyme, functional equivalent thereof or gene encoding such enzyme. In some embodiments, a therapeutic agent may be administered sufficiently prior to administration of the lysosomal enzyme so as to permit modulation (e.g., up-regulation) of the target cell surface receptors to occur, for example, at least about two to about three days, about three to about four days, or about four to about five days before the lysosomal enzyme is administered. For example, in some embodiments, a therapeutic agent may be administered to a patient about 0.25, 0.5, 1, 2, 3, 4, 5, 6, 7, 8, 9, 10, 11 or 12 hours, or 1, 2, 3, 4, 5, 6, 7, 8 days, prior to administration of acid alpha-glucosidase enzyme, modified acid alpha-glucosidase or a functional equivalent thereof.

Administering of a therapeutic agent useful in the disclosed methods may be performed by any suitable route, including administration by inhalation or insufflation (either through the mouth or the nose) or oral, sublingual, buccal, parenteral, topical, subcutaneous, intraperitoneal, intraveneous, intrapleural, intraoccular, intraarterial, rectal administration, or within/on implants, e.g., matrices such as collagen fibers or protein polymers, via cell bombardment, in osmotic pumps, grafts comprising appropriately transformed cells, etc. In particular, the disclosed therapeutic methods and agents are useful for treating cytoplasmic glycogen storage disorder characterized by severe brain involvement without the need for invasive administration techniques directly to brain (e.g., intrathecal administration).

A therapeutic agent, which is capable of enhancing expression of receptors for a lysosomal enzyme, may be administered to the patient as a pharmaceutical composition comprising the therapeutic agent and a pharmaceutically acceptable carrier or excipient. The preparation of a pharmacological composition that contains active ingredients dissolved or dispersed therein is well understood in the art. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that may be employed are water, Ringer's solution and isotonic sodium chloride solution. In addition, solid forms suitable for solution, or suspensions, in liquid prior to use can also be prepared. Formulation also varies according to the route of administration selected (e.g., solution, emulsion, capsule).

Pharmaceutically acceptable carriers can include inert ingredients which do not interact with the β2 agonist, lysosomal enzyme and/or other additional therapeutic agents. These carriers include sterile water, salt solutions (e.g., NaCl), physiological saline, bacteriostatic saline (saline containing about 0.9% benzyl alcohol), phosphate-buffered saline, Hank's solution, Ringer's-lactate saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, dextrose, lactose, trehalose, maltose or galactose, magnesium stearate, talc, silicic acid, viscous paraffin, perfume oil, fatty acid esters, hydroxymethylcellulose and polyvinyl pyrolidone, as well as combinations thereof. The compositions may be mixed with auxiliary agents, e.g., lubricants, preservatives, stabilizers, wetting agents, emulsifiers, salts for influencing osmotic pressure, pH buffers, coloring, flavoring and/or aromatic substances and the like which do not deleteriously react with the active compounds. In addition, the compositions of embodiments described herein may be lyophilized (and then rehydrated) in the presence of such excipients prior to use.

Standard pharmaceutical formulation techniques as known in the art can be employed, such as those described in Remington's Pharmaceutical Sciences, Mack Publishing Company, Easton, Pa. Methods for encapsulating compositions. The composition can be a liquid solution, suspension, emulsion, tablet, pill, capsule, sustained release formulation, or powder. The composition can also be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose or magnesium carbonate. For example, a composition for intravenous administration typically is a solution in a water-soluble carrier, e.g., sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is administered by injection, an ampule of sterile water for injection or saline can be provided so that the ingredients may be mixed prior to administration.

Therapeutic agents of embodiments herein may be administered as neutral compounds or as a salt or ester. Pharmaceutically acceptable salts include those formed with free amino groups such as those derived from hydrochloric, phosphoric, acetic, oxalic or tartaric acids, and those formed with free carboxyl groups such as those derived from sodium, potassium, ammonium, calcium, ferric hydroxides, isopropylamine, triethylamine, 2-ethylamino ethanol, histidine, and procaine. For instance, salts of compounds containing an amine or other basic group can be obtained by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base such as a hydroxide base. Salts of acidic functional groups contain a countercation such as sodium or potassium.

According to some embodiments, a method of treating a glycogen storage disease of embodiments herein may include increasing expression of receptors for acid alpha-glucosidase, or otherwise increasing cell surface density of such receptors, in an individual in need thereof using a therapeutic agent. Representative therapeutic agents capable of inducing such increased expression include growth hormones (e.g., human growth hormone), autocrine glycoproteins (e.g., Follistatin), and β2 agonists. Such therapeutic agents may selectively modulate expression of receptors for the lysosomal enzymes of embodiments herein, (e.g acid alpha-glucosidase). Expression of receptors for acid alpha-glucosidase may also be increased by behaviors, such as exercise. In some embodiments, a β2 agonist is administered to a patient suffering from glycogen storage disease of embodiments herein, wherein administering the β2 agonist results in biochemical correction of the enzyme deficiency in target tissues (e.g. liver) and improved motor function.

Also encompassed by the instant disclosure are methods of increasing efficacy of a glycogen storage disease therapy, e.g., substrate deprivations and small molecule therapies, GAA replacement therapy, including gene therapy (e.g., transfection of cells in a patient with a vector encoding a deficient lysosomal enzyme), or any other form of therapy where the levels of the deficient lysosomal enzyme in a patient are supplemented. For example, these therapies may comprise increasing expression of receptors for a lysosomal enzyme, for example, by administering an effective amount of β2 agonist.

In some aspects, a therapeutic agent capable of increasing expression of receptors for a lysosomal enzyme is administered in combination with a second therapeutic agent or treatment, and in such cases, the therapeutic agents or treatments may be administered concurrently or consecutively in either order. For concurrent administration, the therapeutic agents may be formulated as a single composition or as separate compositions. The optimal method and order of administration of the therapeutic agents capable of increasing expression of a receptor for a lysosomal enzyme and a second therapeutic agent or treatment can be ascertained by those skilled in the art using conventional techniques and in view of the information set out herein.

The disclosed combination therapies may elicit a synergistic therapeutic effect, i.e., an effect greater than the sum of their individual effects or therapeutic outcomes. Measurable therapeutic outcomes are described herein. For example, a synergistic therapeutic effect may be an effect of at least about two-fold greater than the therapeutic effect elicited by a single agent, or the sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single agent, or the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

In some embodiments, for the treatment of a cytoplasmic glycogen storage disorder, a therapeutic agent of embodiments herein may be administered to a patient in combination with a lysosomal enzyme. In some embodiments, a therapeutic agent and lysosomal enzyme may be components of separate pharmaceutical compositions that are mixed together before administration, or that are administered separately. In some embodiments, a therapeutic agent can also be administered simultaneously, without mixing (e.g., by delivery of the β2 agonist on an intravenous line by which the lysosomal enzyme is also administered). In some embodiments, a therapeutic agent may be administered separately (e.g., not admixed), but within a short time frame (e.g., within 24 hours) prior to or subsequent to administration of a lysosomal enzyme. In some embodiments, a therapeutic agent can be administered separately (e.g., not admixed), and without any prior, concurrent, or subsequent administration of a lysosomal enzyme. A synergistic effect may support reduced dosing of ERT when used with a therapeutic agent and a reduced dosing of the therapeutic agent.

For example, in some embodiments, GAA may be administered as a single dose at a single time point, or administered to the patient over the span a several hours (e.g., once every hour, once every two hours, once every three hours, etc.) or over the span of several days (e.g., once a day, once every two days, once every three days, etc.).

Where a combination therapy is used, in some embodiments, administration of a therapeutic agent and the lysosomal enzyme can take place once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or at least once every 1, 2, 3, 4, 5, 6, 7, 8, 9, 10, 11, 12, 13, 14, 15, 16, 17, 18, 19 or 20 weeks, any range of two of these values, or any combination thereof, using single or divided doses of every 60, 48, 36, 24, 12, 8, 6, 4, or 2 hours, or any combination thereof.

In some embodiments, a therapeutic agent (e.g. β2 agonist) is administered prior to, or concurrently with, or shortly thereafter, the lysosomal enzyme, functional equivalent thereof or gene encoding such enzyme. In some embodiments, the therapeutic agent capable of increasing expression of a receptor for a lysosomal enzyme may be administered sufficiently prior to administration of the lysosomal enzyme so as to permit modulation (e.g., up-regulation) of the target cell surface receptors to occur, for example, at least two-three, three-four or four-five days before the lysosomal enzyme is administered. For example, in some embodiments, the β2 agonist may be administered to a patient about 0.25, 0.5, 1, 2, 3, 4, 5, 6, 7, 8, 9, 10, 11 or 12 hours, or 1, 2, 3, 4, 5, 6, 7, 8 days, prior to administration of GAA, modified acid alpha-glucosidase or a functional equivalent thereof.

In some embodiments, the lysosomal enzyme and a therapeutic agent of embodiments herein may be formulated into a composition or medicament for treating the cytoplasmic glycogen storage diseases of embodiments herein. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In some embodiments, a water-soluble carrier suitable for intravenous administration is used.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

In some embodiments, the composition or medicament may be formulated in accordance with the routine procedures as a pharmaceutical composition adapted for administration to human beings. For example, in some embodiments, a composition for intravenous administration typically is a solution in sterile isotonic aqueous buffer. In some embodiments, the composition may also include a solubilizing agent and a local anesthetic to ease pain at the site of the injection. In some embodiments, the ingredients are supplied either separately or mixed together in unit dosage form, for example, as a dry lyophilized powder or water free concentrate in a hermetically sealed container such as an ampule or sachette indicating the quantity of active agent. In some embodiments, where the composition is to be administered by infusion, the composition can be dispensed with an infusion bottle containing sterile pharmaceutical grade water, saline or dextrose/water. In some embodiments, where the composition is administered by injection, an ampule of sterile water for injection or saline can be provided so that the ingredients may be mixed prior to administration.

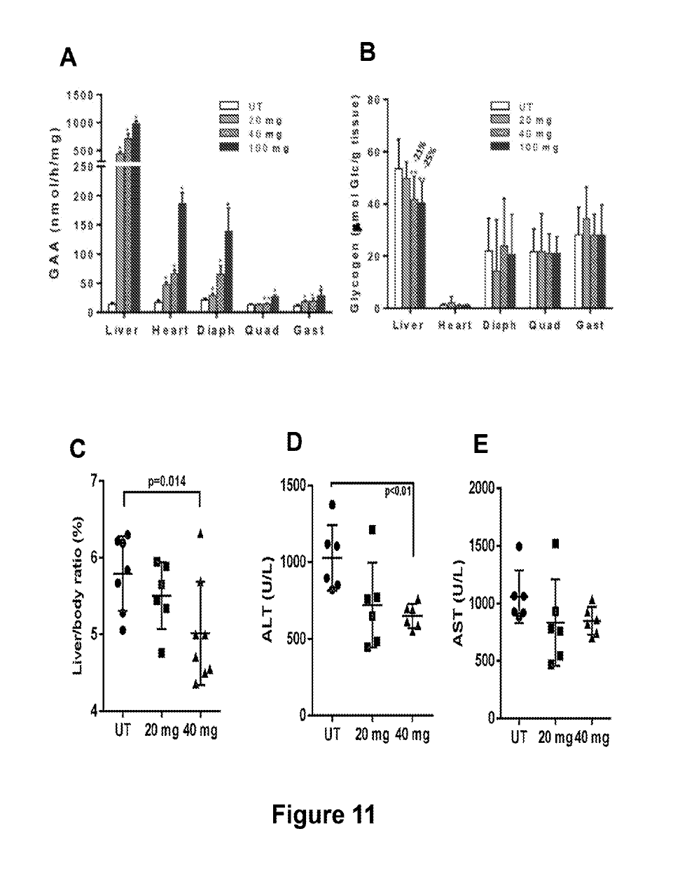

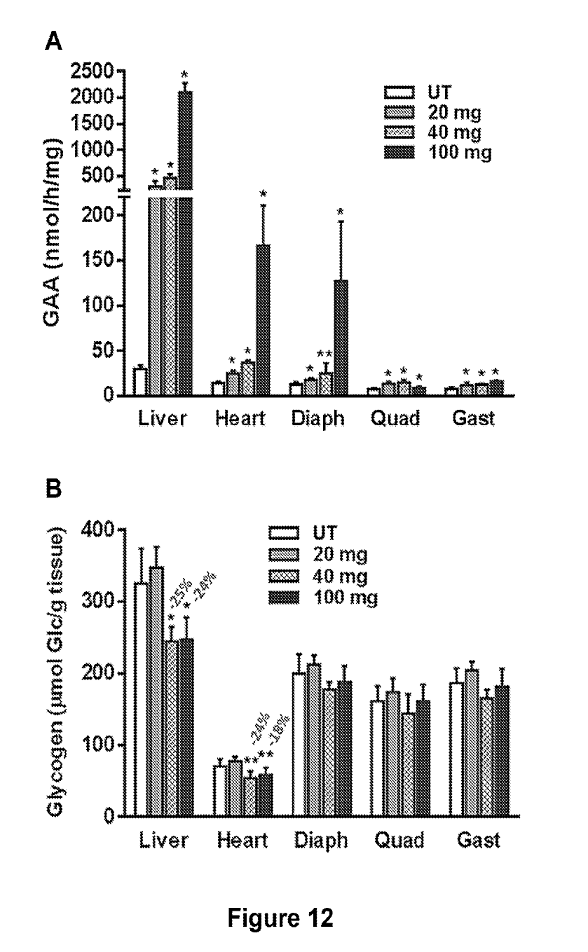

Both GSD III and GSD IV mice generated significantly less anti-GAA antibodies than that reported in the GAA-KO mice, upon a short-term treatment (weekly rhGAA administration for 4 weeks) at a dose of 20 mg/kg, 40 mg/kg, or 100 mg/kg (data not shown). Both the 40 mg- and 100 mg-rhGAA treatment showed a similar impact on liver glycogen in each model, decreasing 24-25% in the GSD III (FIG. 12B) and 21-25% in the IV (FIG. 11B). In addition, both the 40 mg- and 100 mg-rhGAA treatment significantly reduced glycogen contents in the hearts of GSD III mice (FIG. 12B).

While embodiments set forth herein are described in terms of “comprising”, all of the foregoing embodiments also include compositions and methods that consist of only the ingredients or steps recited or consist essentially of the ingredients and steps recited, and optionally additional ingredients or steps that do not materially affect the basic and novel properties of the composition or method.

This disclosure and embodiments illustrating the method and materials used may be further understood by reference to the following non-limiting examples.

### EXAMPLE 1

### rhGAA Reduced Glycogen Accumulation in Cultured Primary Muscle Cells Derived from the GSD IV Mice

Primary myoblast cells were isolated from 7-day-old GSD IV mouse skeletal muscle. Early passage cells were seeded in 10-cm culture dishes with EMEM medium containing 10% FBS. When cells reached 90% confluence, rhGAA was added to the culture medium (final activity =1000 nmol/hr/ml). The cells were harvested 24 hours later to analyze glycogen content and GAA activity. As shown in FIG. 3A, GAA activity increased by 50%, and as shown in FIG. 3B, glycogen content decreased by 24% after the rhGAA treatment.

Reduction of glycogen deposition in GSD IV mouse myoblasts by rhGAA treatment was also confirmed by immunofluorescence staining using a mouse anti-glycogen monoclonal antibody ESG1A9mAb. As shown in FIG. 4, the untreated cells (0 hr) contained heavily stained glycogen particles of various sizes. The reduction of glycogen was obvious at 4 hours (4 hr) and became more evident at 24 hours (24 hr) following the rhGAA treatment.

### EXAMPLE 2

### Characterization of a Mouse Model of GSD IV

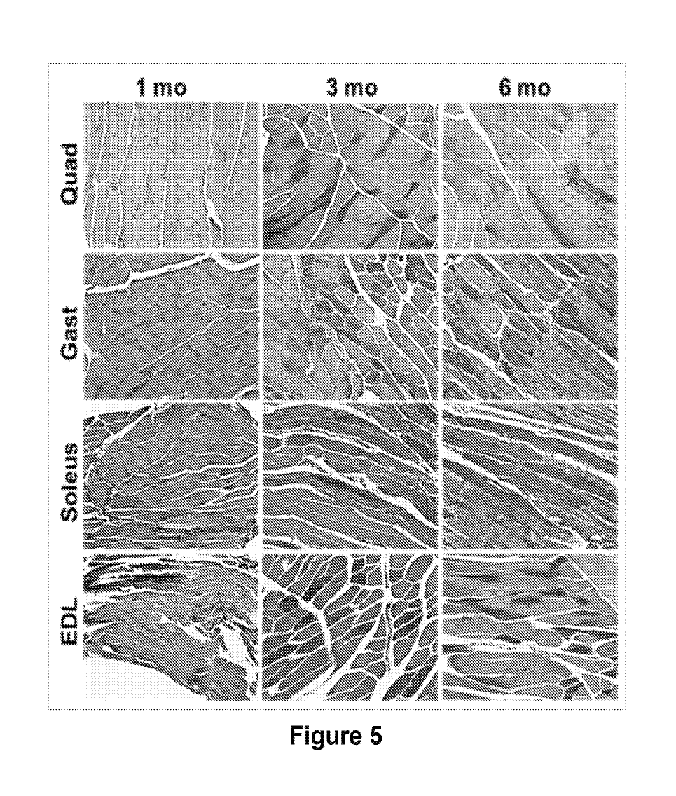

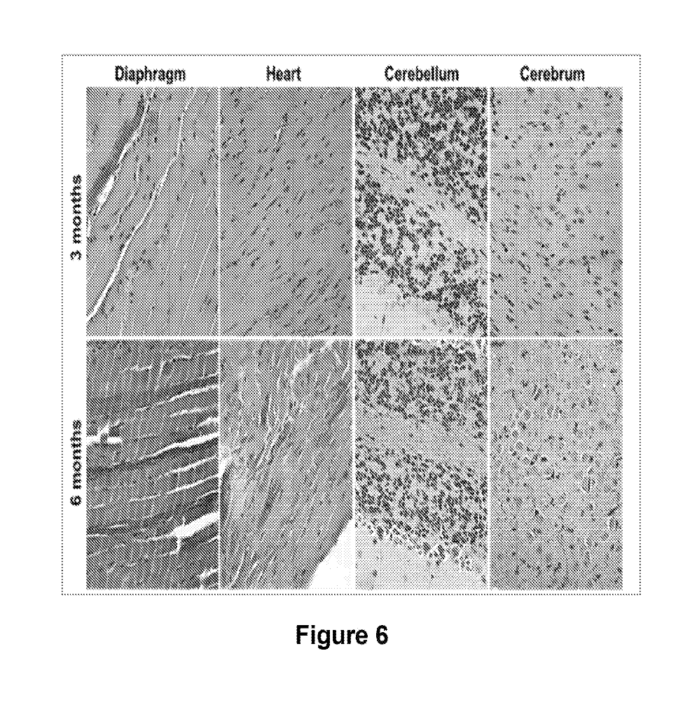

GSD IV is an autosomal recessive disorder caused by deficiency of glycogen branching enzyme (GBE) which results in deposition of less-branched amylopectin-like polysaccharide in muscle, liver, and the CNS. Prior to the present embodied treatment, liver transplantation was the only treatment option for patients with progressive liver fibrosis. A mouse model (Gbel^{ys/ys} model) of GSD IV was obtained from Dr. Craigen and Dr. Akman of Baylor College of Medicine (unpublished). The affected mice (GSD IV mice) carry the Y329S mutation, the most common mutation found in patients with late-onset GSD IV or adult polyglucosan body disease (APBD). PAS stained tissue sections revealed progressive glycogen deposition in skeletal muscles of the Gbe1 mice (FIG. 5). There were less PAS positive particles in diaphragm, heart, and the brain at 3 months of age but became more prevalent at age 6 months (FIG. 6).

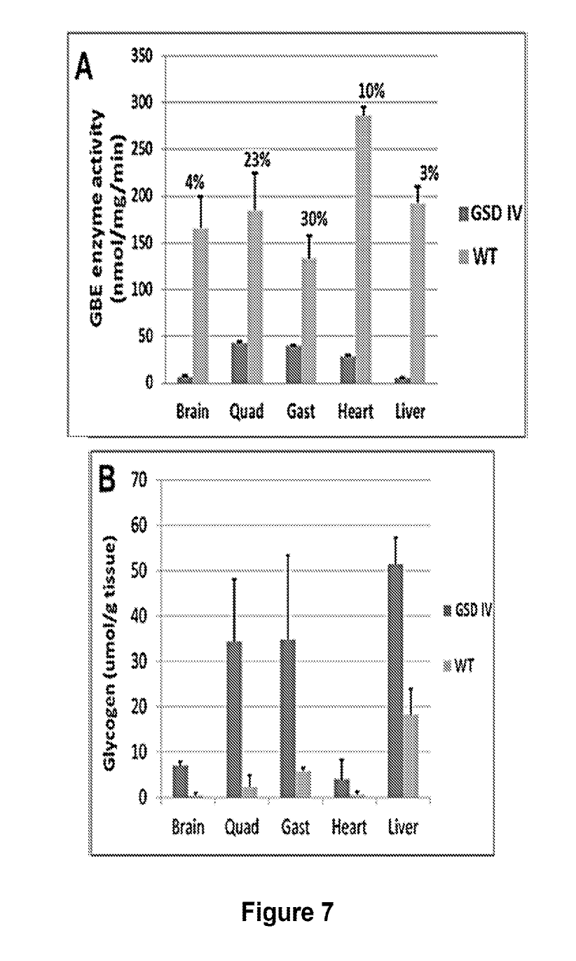

Tissue GBE enzyme activity and glycogen content at age 3 months were compared with age-matched wild-type (WT) mice. As shown in FIG. 7, reduced GBE activity was detected in all tissues of the GSD IV mice, ranging from 3% in the liver to up to 30% in the skeletal muscle (FIG. 7A). Glycogen content was highly elevated in all tissues of the GSD IV mice in comparison with the WT mice (FIG. 7B).

### EXAMPLE 3

### A Modified Enzymatic Method for Measurement of Glycogen Content in Glycogen Storage Disease Type IV

### SUMMARY

Deficiency of glycogen branching enzyme in glycogen storage disease type IV (GSD IV) results in accumulation of less-branched and poorly soluble polysaccharides (polyglucosan bodies) in multiple tissues. Standard enzymatic method, used to quantify glycogen content in GSD IV tissues, causes significant loss of the polysaccharides during preparation of tissue lysates. We report a modified method including an extra boiling step to dissolve the insoluble glycogen, ultimately preserving the glycogen content in tissue homogenates from GSD IV mice. Muscle tissues from wild-type, GSD II and GSD IV mice and GSD III dogs were homogenized in cold water and homogenate of each tissue was divided into two parts. One part was immediately clarified by centrifugation at 4° C. (STD-prep); the other part was boiled for 5 min then centrifuged (Boil-prep) at room temperature. When glycogen was quantified enzymatically in tissue lysates, no significant differences were found between the STD-prep and the Boil-prep for wild-type, GSD II and GSD III muscles. In contrast, glycogen content for GSD IV muscle in the STD-prep was only 11% of that in the Boil-prep, similar to wild-type values. Similar results were observed in other tissues of GSD IV mice and fibroblast cells from a GSD IV patient. This study provides important information for improving disease diagnosis, monitoring disease progression, and evaluating treatment outcomes in both clinical and preclinical clinical settings for GSD IV. This report should be used as an updated protocol in clinical diagnostic laboratories.

### Introduction

In animal cells, glycogen synthesis is primarily catalyzed by two enzymes, glycogen synthase (GS, EC 2.4.1.11), which adds glucose residues to a linear chain, and glycogen branching enzyme (GBE, EC 2.4.1.18), which adds branches to the growing glycogen molecule. Although the majority of glycogen is degraded in the cytoplasm by the combined action of glycogen phosphorylase and glycogen debranching enzyme (GDE, EC 2.4.1.25/EC 3.2.1.33), a small percentage of glycogen is transported to and hydrolyzed in lysosomes by acid α-glucosidase (GAA, EC 3.2.1.20).

Glycogen storage diseases (GSDs) are a group of inherited disorders caused by deficiency of a certain enzyme involved in glycogen synthesis or degradation. While the accumulation of glycogen in liver and muscle tissues is the common consequence of these diseases, the molecular structure and property of glycogen varies between specific GSDs. For example, deficiency of GAA in GSD II causes accumulation of glycogen with normal structure in the lysosomes. In GSD III, loss of GDE enzyme activity hinders further breakdown of glycogen from branching points, resulting in the accumulation of abnormal glycogen with short outer chains. In GSD IV, deficiency of GBE leads to the production of less-branched and poorly soluble polysaccharides (polyglucosan bodies, PB) in all body tissues.

Biochemical quantification of glycogen content is critical for disease diagnosis, disease progression monitoring, and therapeutic outcomes evaluation in both clinical and preclinical settings. An enzymatic method based on homogenization of tissues in cold water followed by Aspergillus niger amyloglucosidase (EC 3.2.1.3) digestion has become widely-used for measuring glycogen content in tissue. In the past decade, our team has had success using this method to quantify glycogen in various tissues from experimental animals with GSD type I, II, or III. Recently, in our work with a mouse model of GSD IV, we found that the measured tissue glycogen contents were at extremely low levels, which contradicts with the observation that strongly PAS-positive PB were present in these tissues. Considering the low solubility of the PB in GSD IV, we speculated that the majority of glycogen was lost during the lysate preparation. Here we describe a modified enzymatic method for glycogen quantification in GSD IV.

### Materials and Methods

### Animal Tissues

Muscle tissues were obtained from 3-month-old GAA knockout (GSD II) mice (Raben et al., 1998) and from 4-month-old GSD Ma dogs. GSD IV (Gbelys/ys) mice were euthanized at age of 3 months following overnight fasting for collection of tissues. Muscle tissues from 3-month-old wild-type (C57BL/6) mice were used as controls. Fresh tissues were fixed in 10% neutral buffered formalin for PAS staining or frozen in −80° C. freezer until use. All animal experiments were approved by the Institutional Animal Care & Use Committee at Duke University and were in accordance with the National Institutes of Health guidelines.

### Tissue Lysate Preparation

Frozen tissues (50-100 mg) were homogenized in ice-cold de-ionized water (20 ml water/g tissue) and sonicated three times for 15 seconds with 30-second intervals between pulses, using a Misonix XL2020 ultrasonicator. Homogenate of each tissue was divided into two parts and processed separately: one part was immediately clarified by centrifugation at 4° C. (STD-prep); the other part was boiled for 5 min then centrifuged at room temperature (Boil-prep).

### Cell Culture and Cell Lysates

Fibroblasts derived from skin biopsies of a patient with GSD II and one with GSD IV were harvested after 3 days in culture in 10-cm plates. The cell pellet from each plate was resuspended in 300 μl cold water and sonicated three times. The STD-prep and Boil-prep cell lysates were then prepared as described above. Protein concentration of the STD-prep was determined using BCA method.

### Glycogen Content Measurement

Glycogen contents in the tissue and cell lysates (both the STD-prep and the Boil-prep) were assayed.

### Statistical Analysis of Glycogen Content

The significance of differences between the STD-prep and Boil-prep of the same group of samples was assessed using two-tailed, paired student T-test. Mean ±standard deviation were shown.

### Results

### Glycogen Staining and Quantitation in Skeletal Muscles From Wild-Type and GSD Animals

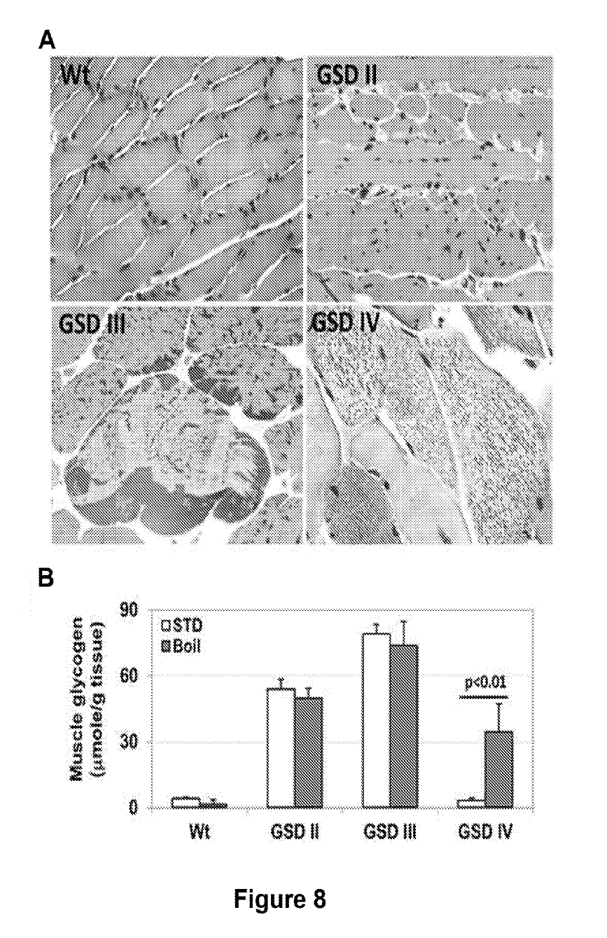

PAS staining of glycogen revealed no visible PAS-positive materials in wild-type (Wt) mice. In GSD II mice, glycogen-filled lysosomes of various sizes were scattered throughout the tissue; in GSD III dogs, filamentous glycogen aggregates and large pools of glycogen were seen; in GSD IV mice, granular glycogen particles were observed in most myocytes (FIG. 8A).

When glycogen was quantified in tissue lysates, no significant differences were found between the STD-prep and the Boil-prep for wild-type (Wt), GSD II and GSD III muscles (FIG. 8B). In contrast, the GSD IV muscle showed a very low level of glycogen in the STD-prep lysates (3.17±1.15 μmol glucose/g tissue), similar to that of wild-type muscle, while the Boil-prep showed a markedly higher level (34.5±12.7), indicating significant loss of glycogen in the STD-prep lysates (FIG. 8B).

Glycogen Staining and Quantitation in other Tissues from GSD IV Mice

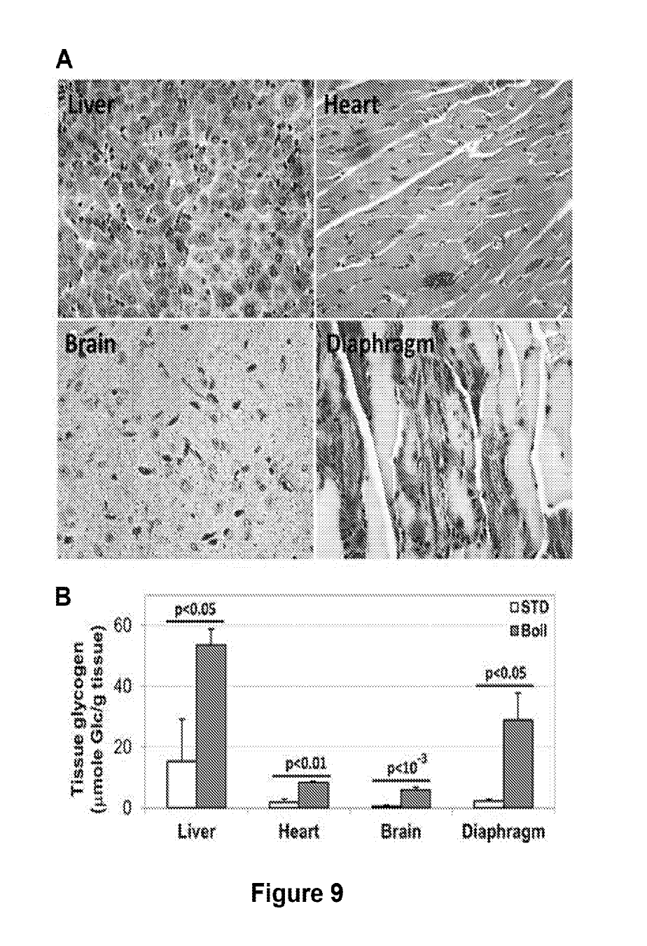

PAS staining of glycogen was also performed on other tissues of GSD IV mice at age 3 months. As shown in FIG. 9A, most hepatocytes were loaded with glycogen (fasted); the diaphragm has similar glycogen accumulation pattern as the gastrocnemius muscle; clusters of glycogen particles were occasionally found in the heart; PAS-positive granules were clearly present in the brain (cerebrum). Glycogen quantitation showed significantly lower glycogen contents in the STD-preps than in the Boil-preps for all the tissues (FIG. 9B). Glycogen content in the STD-prep was 28% of that in the Boil-prep for liver (fasted), and was 21% for heart, 8% for both brain and diaphragm (FIG. 9B).

Glycogen Quantitation in Fibroblasts from Patients with GSD II and IV

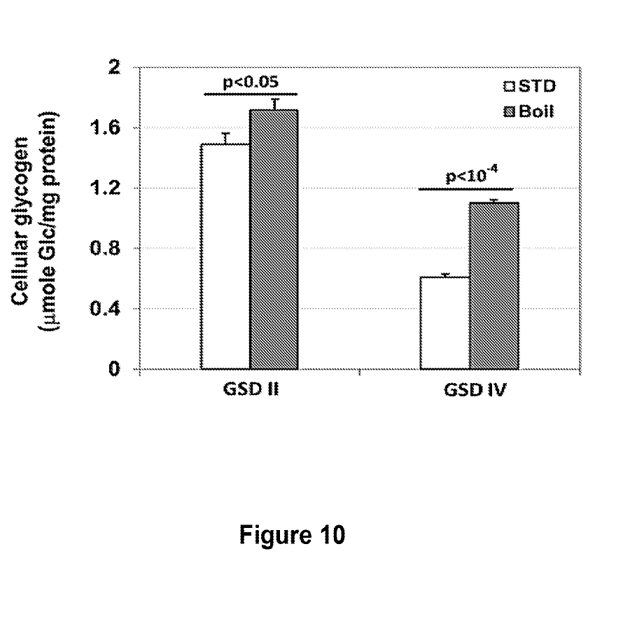

In cultured human patient skin fibroblasts, the STD-prep of the GSD IV cells presented 50% less glycogen than the Boil-prep; the Boil-prep of GSD II cells presented 10% more glycogen than the STD-prep (FIG. 10).

### Discussion

Mutations in the Gbe1 gene cause a complete or partial loss of GBE activity in GSD IV, which leads to an increase in the ratio of GS to GBE, a critical determinant of PB formation during the process of glycogen synthesis. The Y329S is the most common mutation found in Jewish families of Ashkenazi ancestry with adult onset GSD IV, also referred to as adult polyglucosan body disease. Recently we obtained a new mouse model of GSD IV (Gbe1ys/ys mice) carrying the knock-in Y329S mutation. The residual enzyme activity in the affected mice was approximately 24-30% of wild-type value in skeletal muscles, 10% in heart, and less than 5% in liver and brain (data not shown). PAS staining showed significant PB accumulation in all these tissues.

In a standard enzymatic method for glycogen quantitation, tissue homogenization in cold water or buffer followed by an immediate centrifugation has been a widely used procedure for its simplicity, sensitivity, and ability to analyze other metabolites and enzyme activities in the same homogenate. But this procedure is not suitable for GSD IV glycogen measurement due to the heavy loss of insoluble glycogen during sample preparation. In this study, we described a modified method that includes an extra boiling step prior to centrifugation of tissue homogenates to dissolve the insoluble glycogen in GSD IV. To determine the length of boiling time needed for complete glycogen dissolution, we quantified glycogen after boiling the homogenates (150-300 μl) 3, 5, 10, and 15 minutes and saw no difference among all the time points (data not shown). This method is likely also applicable to Lafora disease, a related polyglucosan body disease caused by mutations in EPM2A or EPM2B, but this needs to be verified by experiments . Another more tedious and less sensitive method involving boiling tissue homogenate in KOH followed by ethanol-precipitation of glycogen prior to the amyloglucosidase digestion is also suitable for determining glycogen content in GSD IV, but this procedure requires larger size of tissues, which limits its clinical application.

This study provides an improved protocol for quantifying the insoluble glycogen in GSD IV without the need of glycogen isolation prior to the enzyme digestion. More importantly, the modified method allows determination of glycogen content in very small biopsy samples, which is extremely useful for clinical diagnostic laboratories. Validation with sufficient numbers of patient samples and normal controls will be necessary before applying this method to clinical diagnosis.

### EXAMPLE 4

### Alglucosidase Alfa Enzyme Replacement Therapy as a Therapeutic Approach for GSD IV

Methods: A short-term study was conducted to determine the minimum effective dose (MED) of rhGAA treatment with 3 dosages: 20 mg/kg (human equivalent dose, n=6), 40 mg/kg (n=9), and 100 mg/kg (n=8). Male GSD IV mice received weekly intravenous injections of rhGAA were conducted for 4 week starting at age of 10 weeks. A group of age-matched untreated mice (n=8) were used as controls. To prevent anaphylactic reactions, the animals were administered 25 mg/kg diphenhydramine (i.p.) 10-15 min prior to enzyme administration. All mice were sacrificed 48 hours after the last injection following overnight fasting. Fresh tissues were immediately frozen and stored at −80° C. until use for GAA activity and glycogen content analyses. Protein concentration was measured using BCA method.

Results: As shown in FIG. 11A, significant increase in GAA activity was observed in tissues of GAA-treated mice in a dose dependent manner. The greatest increase was found in liver, which had 29, 48, and 67 folds increase over untreated controls at the 3 doses from low to high, respectively. GAA activity in heart had a 1.7-fold increase in the 20 mg/kg dose group and 2.8-fold increase in the 40 mg/kg group. In quadriceps the increase in GAA activity was negligible at either dosage, while uptake by gastrocnemius was slightly more, with less than 1-fold increase of GAA activity in either treated group. Diaphragm had the highest GAA activity increase among the skeletal muscles tested, with increases of GAA activity similar to those in heart by the 40 mg/kg treatment. Enzyme uptake was less efficient in skeletal muscles as the GAA activity was increased by 1.6 folds at 100 mg/kg. Glycogen contents were significantly reduced only in liver of the 40 mg/kg (−21%) and 100 mg/kg (−25%) groups, not in any skeletal muscle (FIG. 11B). The low level of glycogen in heart of this GSD IV mouse model makes it difficult to draw a conclusion for this tissue (FIG. 11B). The 20 mg/kg GAA treatment failed to reduce glycogen in any tissue (FIG. 11B).

Consistent with reduced liver glycogen accumulation, the 40 mg/kg rhGAA treatment lowered liver/body weight ratio from 5.8±0.2% to 5.0±0.2% (p<0.05; FIG. 11C), and reduced plasma alanine aminotransferase (ALT) from 1029±87 U/L to 650±32 U/L (p<0.01; FIG. 11D) and aspartate aminotransferase (AST) from 1059±93 U/L to 849±50 U/L (p=0.074; FIG. 11E), indicating alleviation of hepatomegaly and liver damage.

Discussion: Manose-6-phosphate receptor (M6PR) mediated ERT with rhGAA is an FDA approved therapy for Pompe disease. The pattern of rhGAA uptake by tissues of GSD IV mice (FIG. 11A) was similar to that observed in Pompe disease mice. The high GAA activity in liver and low activity in muscles following rhGAA treatment correlated well with the relative abundances of the M6PR in the two types of tissues. Even though the 20 mg/kg treatments led to significantly higher GAA activities in liver, the reduction of glycogen accumulation was not significant (FIG. 11B). This suggests that the insolubility of GSD IV glycogen makes it highly resistant to rhGAA digestion. Thus, it is not surprising to see the lack of effectiveness in skeletal muscles, which showed low uptake of rhGAA after treatment (FIG. 11A, B). Our interpretation for the reduction of liver glycogen in GSD IV mice by the high-does rhGAA treatment is that digestion of the insoluble GSD IV glycogen in lysosomes requires highly elevated rhGAA activity; clearance of lysosomal glycogen promotes glycogen trafficking into lysosomes, and thus reduces the overall glycogen accumulation. However, it is also possible that the excessive amount of rhGAA in lysosomes led to leakage of the enzyme into the cytoplasm and directly degraded the accumulated glycogen, even though the activity of GAA in the neutral pH environment is much lower than that in the acidic lysosome interior.

The typical clinical presentation of patients with hepatic GSD IV, such as hepatomegaly and elevation of liver enzymes caused by liver damage, was also observed in this GSD IV mouse model (FIGS. 11C, 11D, 11E). The biochemical correction of liver glycogen accumulation by the 40 mg/kg rhGAA treatment was accompanied by the attenuation of clinical liver symptoms, as indicated by the reduction of liver size (as determined by the liver/body weight ratio) and of liver enzymes in serum. Moreover, one apparent advantage of treating GSD IV with rhGAA is that, as patients express normal level of GAA, the therapeutic protein is unlikely to induce severe immune responses, which have been a major obstacle in treatment of Pompe disease. This data suggests that rhGAA could be a potential therapy for GSD IV and possibly other cytoplasmic GSDs.

### EXAMPLE 5

### Investigation of Long-Term Treatment Efficacy with the Minimum Effective Dose (40 mg/kg) of rhGAA in GSD IV Mice, with or without the Adjunctive Therapy with Clenbuterol

Clenbuterol, a β2 agonist, will be used in this study. Unlike in GAA-KO mice where glycogen is accumulated in lysosomes, treatment for GSD IV mice would require a longer course of treatment to demonstrate efficacy in reducing the cytoplasmic glycogen accumulation. All experiments will last for 3 months. There will be 4 groups, n=8-10 mice per group:

| GROUP | NAME | TREATMENT |
|---|---|---|
| Group 1 | Mock-treatment group | weekly saline I.V. injection |
| | | for 4 weeks |
| Group 2* | rhGAA only | weekly I.V. at MED |
| Group 3* | Clenbuterol treatment only | Clenbuterol administered ad |
| | | libitum in drinking water at |
| | | 30 μg/ml |
| Group 4* | Clenbuterol + rhGAA | rhGAA administered weekly |
| | | I.V. at MED |

*For each mouse, pretreatment with 15-25 mg/kg diphenhydramine by i.p. injection will be performed 10-15 min prior to rhGAA administration to prevent anaphylactic reactions.

All treatment will start at age of 3 months. Urine will be collected at ages of 3 and 6 months for testing urinary Hex4, a biomarker for Pompe disease, by stable isotope-dilution electrospray tandem mass spectrometry. Blood will be collected months from age 3 months to test anti-GAA antibody titers. Behavioral and muscle function will be tested at ages 3, 4.5, and 6 months, to assess reversal of neuromuscular involvement by treadmill, Rota-rod performance, wire-hang, and grip strength tests. All mice will be euthanized at age of 6 months for collection of 1) tissues including liver, heart, skeletal muscles, diaphragm, and the brain for histological and biochemical analysis.

### EXAMPLE 6

### Generation and Characterization of a Mouse Model of GSD IIIc

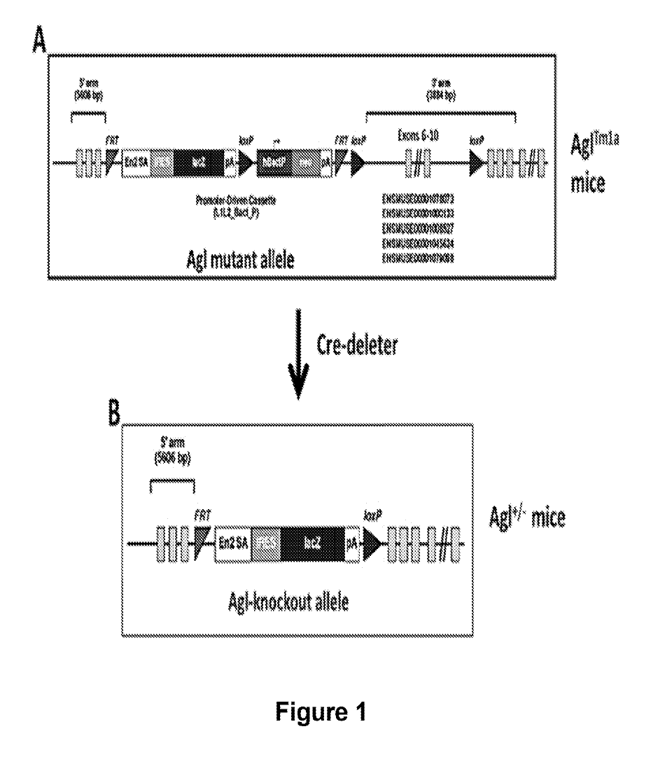

Heterozygous AGL mutant mice (Ag1Tm1a) carrying a mutant Agl allele (FIG. 1A) were purchased form The European Mouse Mutant Archive (EMMA). We have cross-bred this mouse line with a Cre deleter strain (CMV-Cre mice) to convert the mutant allele into an Agl-KO allele by deleting the Agl gene Exons 6-10 and the neo expression cassette (FIG. 1B). We have successfully crossed Agl+/− mice to generate homozygous Agl−/− (GSD III) mice. Once sufficient GSD III mice become available, we will characterize this model by analyzing these mice at different ages (1, 3, 6, and 9 months) for 1) tissue histology and glycogen contents; 2) muscle function performance by treadmill, Rota-rod performance, wire-hang, and grip strength tests; 3) urinary Hex4 levels.

### EXAMPLE 7

### Alglucosidase Alfa Enzyme Replacement Therapy as a Therapeutic Approach for GSD III

Methods: GSD III mice were used to test 3 dosages to determine the minimum effective dose (MED) of rhGAA treatment: 20 mg/kg (n=6), 40 mg/kg (n=5), and 100 mg/kg (n=7). Weekly intravenous injections of rhGAA were conducted for 4 week starting at age of 10 weeks. A group of age-matched untreated mice (n=8) were used as controls. To prevent anaphylactic reactions, the animals were administered 25 mg/kg diphenhydramine (i.p.) 10 -15 min prior to enzyme administration. All mice were sacrificed 48 hours after the last injection following overnight fasting. Fresh tissues were immediately frozen and stored at −80° C. until use for GAA activity and glycogen content analyses. Protein concentration was measured using BCA method.

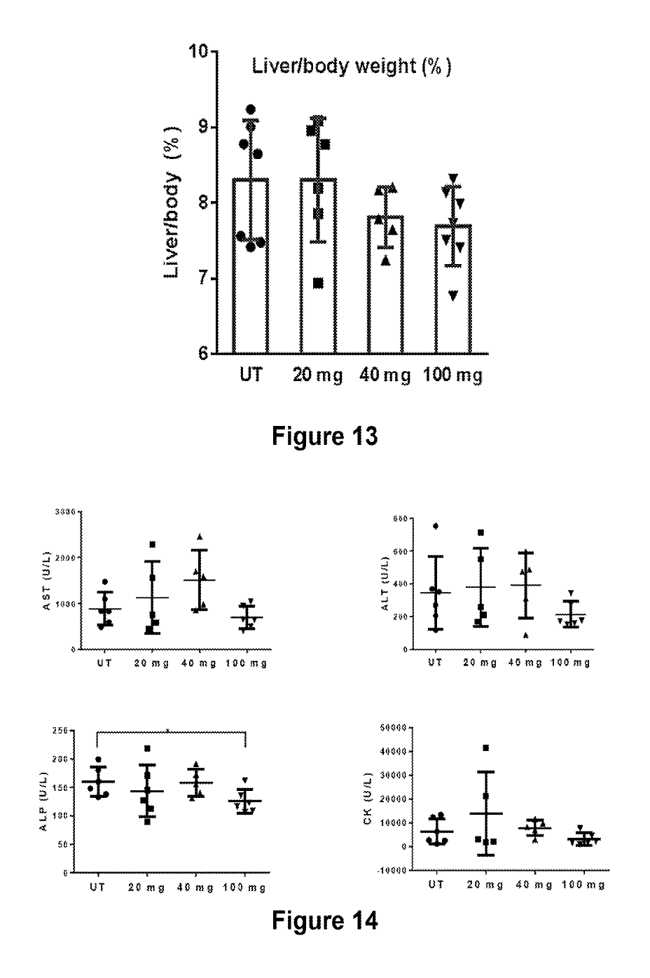

Results: GAA enzyme uptake was similar as seen in GSD IV mice: GAA activity in liver>heart>diaphragm>leg muscles (FIG. 12A). Both the 40 mg and 100 mg treatments significantly reduced glycogen contents to a similar level in liver of GSD III mice (FIG. 12B), accompanied by reduced ratio of liver/body weight (FIG. 13). In addition, both the 40-mg and 100-mg treatments significantly reduced glycogen contents in heart of GSD III mice (FIG. 12B). There was no significant change in glycogen content in skeletal muscles in the 40-mg and 100-mg treatment groups. The 20-mg treatment did not affect glycogen content in any tissues of the GSD III mice. There was also a reduction in plasma alanine aminotransferase (ALT), alkaline phosphatase (ALP), aspartate aminotransferase (AST), and creatine kinase (CK), indicating alleviation of hepatomegaly and liver damage. (FIG. 14) A long-term (up to 3 months) treatment efficacy with 40 mg/kg rhGAA will be evaluated in GSD III mice as outlined below.

### EXAMPLE 8

### Long Term Efficacy with rhGAA at the Minimum Effective Dose (40 mg/kg) of rhGAA Treatment in GSD III Mice

Specific Aim: To investigate long-term treatment efficacy with rhGAA at a dose of 40 mg/kg in GSD III mice. Development of antibody response against human protein (rhGAA) in GSD III mice will reduce efficacy of Myozyme treatment. In this study Methotrexate (MTX) will be used to induce immune tolerance to rhGAA treatment in GSD III mice. There will be 2 groups, n=8 mice per group:
Group 1. Untreated group—no treatment controls;
Group 2. rhGAA treatment group*—weekly intravenous (I.V.) injection with rhGAA at 40 mg/kg for 12 weeks. Methotrexate at a dose of 10 mg/kg will be administered intraperitoneally (LP.) at 0, 24 and 48 hour following the initial three weekly rhGAA administrations for each mouse, pretreatment with 15-25 mg/kg diphenhydramine by I.P. injection will be performed 10-15 min prior to rhGAA administration to prevent anaphylactic reactions.

All treatment will start at age of 8 weeks. Urine will be collected at ages of 8 and 20 weeks for testing urinary Hex4, a biomarker for Pompe disease, by stable isotope-dilution electrospray tandem mass spectrometry as previously described. Blood will be collected every 4 weeks to test anti-GAA antibody titers. All mice will be euthanized at age of 20 weeks. Weight of liver and whole body will be measured. Tissues including liver, heart, skeletal muscles, and diaphragm will be collected for histological and biochemical analysis. Plasma will be collected for analysis of liver enzyme activities.

### EXAMPLE 9

### Evaluation of the Long-Term Treatment Efficacy with the Minimum Effective Dose (40 mg/kg) of rhGAA in GSD III Mice, with or without the Adjunctive Therapy with Clenbuterol

Clenbuterol, a β2 agonist, will be used in this study. All experiments will last for 3 months. There will be 4 groups, n=8-10 mice per group:

| GROUP | NAME | TREATMENT |
|---|---|---|
| Group 1 | Mock-treatment group | weekly saline I.V. injection |
| | | for 4 weeks |
| Group 2* | rhGAA only | weekly I.V. at MED |
| Group 3* | Clenbuterol treatment only | Clenbuterol administered ad |
| | | libitum in drinking water at |
| | | 30 μg/ml |
| Group 4* | Clenbuterol + rhGAA | rhGAA administered weekly |
| | | I.V. at MED |

*For each mouse, pretreatment with 15-25 mg/kg diphenhydramine by i.p. injection will be performed 10-15 min prior to rhGAA administration to prevent anaphylactic reactions.

All treatment will start at age of 3 months. Urine will be collected at ages of 3 and 6 months for testing urinary Hex4, a biomarker for Pompe disease, by stable isotope-dilution electrospray tandem mass spectrometry. Blood will be collected months from age 3 months to test anti-GAA antibody titers. Behavioral and muscle function will be tested at ages 3, 4.5, and 6 months, to assess reversal of neuromuscular involvement by treadmill, Rota-rod performance, wire-hang, and grip strength tests. All mice will be euthanized at age of 6 months for collection of tissues including liver, heart, skeletal muscles, diaphragm, and the brain for histological and biochemical analysis.

### EXAMPLE 10

### Use of Alglucosidase Alfa Enzyme Replacement Therapy for Conditions Associated with PRKAG2 Mutations

This example focuses on a patient initially diagnosed with Pompe disease and started on ERT with alglucosidase alfa, which improved his condition. However, over the course of the therapy, the patient began to develop inconsistent symptoms that led his physicians to question the diagnosis. Through further medical tests, the patient was diagnosed as a carrier of Pompe disease, in addition to carrying a PRKAG2 pathogenic gene mutation. This example further outlines the improvement that the patient showed while on ERT treatment, the decline to his condition when his infusions were discontinued due to his updated diagnosis, and the significant positive response when ERT was reinitiated. This example provides several key messages: 1) the importance of confirming the diagnosis of Pompe disease via gene sequencing before ERT initiation, 2) the potential of GAA as a treatment approach for cytoplasmic GSDs such as PRKAG2, and 3) the expansion of the PRKAG2 phenotype depicting the first report of a case with myopathy and no obvious cardiac involvement.

A male patient was born by caesarian section at 38 weeks gestation as a result of the nuchal cord being wrapped around his neck. At age 2½ months, the patient was noted to have hypotonia and generalized muscle weakness. He was areflexic and had feeding difficulties. At age 4 months, the patient began developing severe lower respiratory infections which led to frequent admissions to the hospital. Labs showed a mild increase in creatinine kinase (CK) at 197 IU/L (normal range: 38-174 IU/L) while other labs including ALT (15 IU/L; normal range: <45 IU/L) and AST (47 IU/L; normal range: 9-80 IU/L) were normal. Following numerous recurrent pneumonias, and the history of muscle weakness, the patient's physicians raised Pompe disease as a potential diagnosis. Blood GAA enzyme testing revealed a deficiency (4.81 units versus 18.66 units in the control sample). An ECHO revealed mild hypertrophy of the interventricular septum (IVS) and a normal sized left ventricular posterior wall (LVPW) with a normal left ventricular mass. The ECG showed that the ventricular forces were normal with a SR of 146/min and a PR interval of 0.10. Given the early findings of hypotonia and the low GAA enzyme activity, a diagnosis of non-classic infantile Pompe disease was made. The patient was initiated on ERT with alglucosdase alfa at a dose of 20 mg/kg every 2 weeks at age 11 months.

At age 11 months, the patient started ERT. He was belly crawling asymmetrically and was unable to achieve sitting from a prone or supine position. Due to weakness in his neck and trunk flexors, the patient consistently sat with his trunk completely collapsed in kyphotic position and his head propped up in capital extension in a chin poke position. This weakness also caused the patient to struggle with clearing secretions while coughing. Three months after the initiation of ERT, the patient's gross motor abilities as assessed by his physical therapist began improving and he achieved new milestones. He was able to crawl more efficiently, to achieve sitting independently from a prone or supine position, and to pull himself up into a standing posture without aid. The patient's level of endurance also improved which allowed him to be more active. At age 29 months, the patient was able to walk independently with an age appropriate gait pattern and to climb small steps as well as jump off them without support. He was also able to transition independently into and out of any position, which helped him participate more fully in activities appropriate for his age.

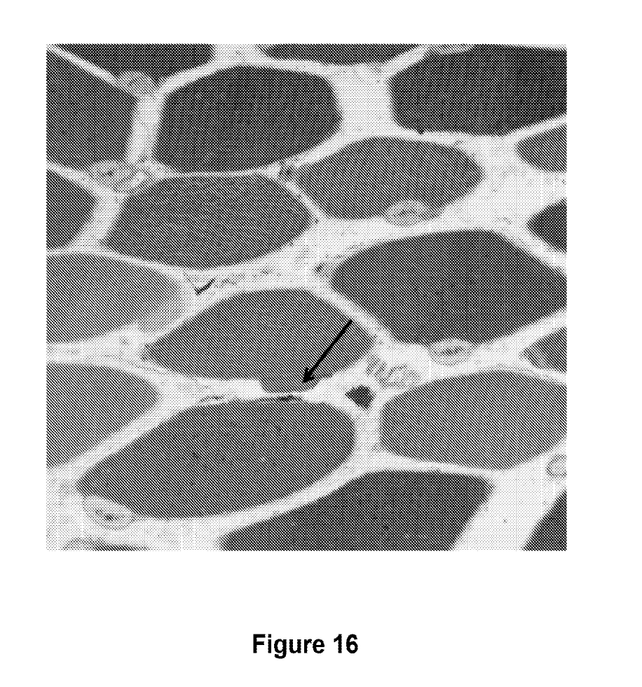

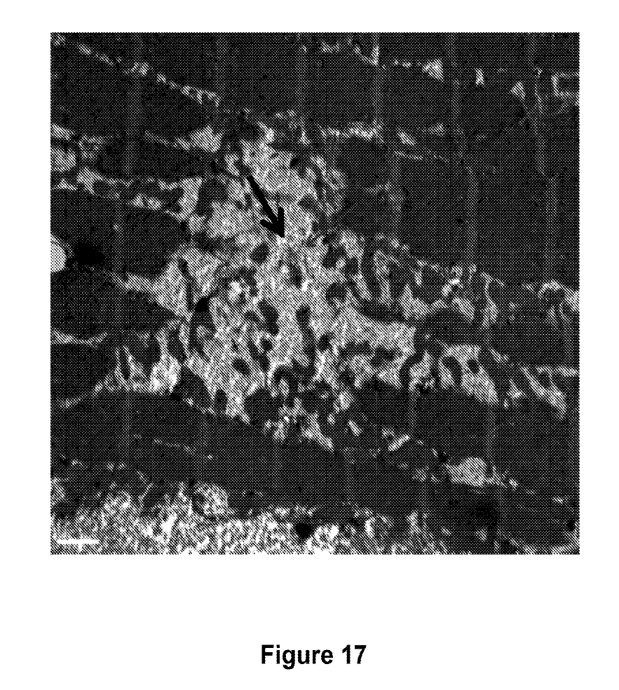

Interestingly at age 13 months, one month after the start of ERT, the patient began to have febrile and non-febrile seizures. An EEG completed at age 14 months revealed epileptic activity. He exhibited tremors of the head and extremities at intermittent intervals with the tremors growing worse upon awakening and while in the motion of reaching. The patient was evaluated by a neurologist and was noted to have an intention tremor, titubation, ataxia, and very mild hypotonia. At age 4 years, he developed complex partial seizures compounded by a 2-3 day period where he was completely floppy and weak, often unable to lift his head off the pillow. Based on these events, the neurologist diagnosed the patient with a complex genetic epilepsy syndrome. As the patient's medical history was somewhat unusual for one with infantile Pompe disease, further evaluation was initiated to determine if he had another diagnosis in addition to Pompe disease to explain these findings or if the initial diagnosis was incorrect. At age 2.5 years (30 months) GAA enzyme activity was done on skin fibroblasts, which was suggestive of a carrier status (50.7 nmol/h/mg with a control range of 45-180 nmol/h/mg and a patient range of 0-20 nmol/h/mg). Sequencing of the GAA gene found the patient to be heterozygous for the common splice site mutation c.-32-13 T/G, a pathogenic mutation among patients with the adult form of Pompe disease. No other mutation was identified; these findings were consistent with carrier status. ERT was discontinued for the patient at age 33 months after 22 months on ERT. A quadriceps muscle biopsy was obtained at age 44 months showed cytoplasmic glycogen, suggestive of a non-lysosomal glycogen storage disease (FIGS. 16 and 17). Muscle acid alpha glucosidase activity tested in the low normal range suggestive of carrier status; phosphorylase and phosphorylase kinase activities measured in normal ranges. Further work up included a mitochondrial myopathy enzyme panel and a mitochondrial respiratory chain panel which were normal and a glycogen storage disease sequencing panel (GCTS Pathology, London, UK) which showed that the patient had a pathogenic mutation in PRKAG2, c.298G>A p. (Gly100Ser), which had been reported previously in cases of PRKAG2 (Table 3 below). As PRKAG2 is inherited in an autosomal dominant manner, the family history was taken again with a focus on the patient's maternal cousin once removed who suddenly died at age 29 due to a cardiac event. The hospital records indicated that the EKG taken at the day of his death indicated a normal sinus rhythm with normal repolarization, normal PR and QTc with no Brugada pattern. There was mild ST segment depression in the inferior leads.

TABLE 3
Literature review of PRKAG2 cases with symptoms and genotype

| | Number | | | | |
|---|---|---|---|---|---|
| Reference | of Cases | Ages | Resolution | Symptoms Shown | Genotype |
| Zhang et al. | 9 cases | 16-49 | 3 deceased | Wolff-Parkinson-White | PRKAG2 |
| (2013) | | years | | syndrome, conduction systen | G100S |
| | | | | disease, and/or hypertrophic | missense |
| | | | | carthomyopathy | mutation* |
| Laforet et | 1 case | 38 | Not | Sinusal bradycardia, high | PRKAG2 |
| al. (2006) | | years | deceased | degree of ventricular block, | S548P |
| | | | | hypertrophic cardiomyopathy | missense |
| | | | | | mutation |
| Burwinkel | 1 case | 34 days | deceased | Fetal bradycardia, | PRKAG2 Het |
| et al. (2005) | | | | preventricular hypertrophy, | K531Qh |
| | | | | cardiomegaly, severe | mutation |
| | | | | hypertrophic cardiomyopathy | |
| Buhrer et al. | 1 case | 21 days | deceased | Bradycardia, atrial and | PRKAG2 Het |
| (2003) | | | | biventricular hypertrophy, | R531Qh |
| | | | | pericardial effusion | mutation |
| Arad et al. | 6 cases | 19-55 | unknown | Cardiac hypertrophy, | PRKAG2 |
| (2002) | | years | | ventricular pre-excitation | R302G (4 |
| | | | | (Wolff-Parkinson-White | cases), |
| | | | | Syndrome), progressive | PRKAG2 |
| | | | | dysfunction of the conduction | T400N (1 |
| | | | | system | (case), |
| | | | | | PRKAG2 |
| | | | | | N4881 (1 case) |
| | | | | | missense |
| | | | | | mutation |
| Gollob et al. | 4 cases | 8-41 | unknown | Wolff-Parkinson-White | PRKAG2 |
| (2001) | | years | | Syndrome, ventricular | R531G |
| | | | | preexcitation, early onset of | missense |
| | | | | atrial fibrillation and | mutation |
| | | | | conduction disease | |
| Relgado et | 2 cases | Birth-2 | deceased | Cardiomegaly, bradycardia, | PRKAG2 Het |
| al (1999) | | months | | cardiorespiratory problems | R531Qh |
| | | | | | mutation |
| Zhang et al. | Zebrafish | — | — | Thicker heart wall, increased | PRKAG2 |
| (2014) | cases | | | glycogen storage in heart wall | G100S |
| | | | | | missense |
| | | | | | mutation* |

*denotes same mutation as patient in case report (G100S)

During the period of 14 months without ERT (age 33 months to age 47 months), the patient was followed closely and clinical decline was noted. The physical therapist observed that the patient was struggling to participate in physical therapy (PT) sessions, which had been easily managed previously while on ERT. He was falling frequently. The patient, who had previously been very interactive during sessions, tended to lie on the floor or on the equipment with significant lack of energy. In addition, there was a decline in his speech and communication; the patient often mumbled and refused to answer his therapist when prompted.

Due to the patient's regressions, past clinical benefit, family request, support from his physicians, and past literature revealing a potential role of alglucosidase alpha in individuals with a cytoplasmic GSD, the patient received IV alglucosidase alfa treatment for 6 months on a trial basis with close follow up after the initiation of therapy (started at age 47 months). Baseline assessments were done which included tests for AST (44 IU/L; normal range: <45 IU/L), ALT (20 IU/L; normal range: 9-80 IU/L), along with PT measures looking at muscle strength and function measures. After five months of ERT, the patient has shown significant improvement according to his neurological, occupational therapy, and physical therapy reports. The patient no longer exhibited myopathic faces or ptosis, his tenting of his upper lip had improved, and he had more facial expression than before. Upon examination, the patient has developed more defined calf muscles, along with an improvement of his strength and power. According to his neurologist (PH), in addition to improvement in strength, the patient's seizures appear to be better controlled since the reinitiation of ERT with alglucosidase alfa; however, this is difficult to understand given that ERT does not cross the blood brain barrier. He has grown physically stronger and has not had episodes in which he lacks energy for consecutive days, becomes completely floppy, and is unable to hold his head up properly following a seizure.

His PT reports indicated that he was learning new motor skills or improving his current skills, which were now well within the average for motor tasks (Table 4 below). The patient's physical therapy reports from before ERT reinitation and post-5 months have recorded significant improvements based on assessment with Movement ABC (MABC) which provides quantitative and qualitative data about a child's performance of age-appropriate tasks within 3 subsections: 1) Manual Dexterity, 2) Ball Skills, and 3) Static and Dynamic Balance. At baseline, according to the MABC assessment, the patient achieved a Total Test Score of 68 ranking him in the 25^{th} percentile. Five months after reinitiation of ERT, the patient achieved a Total Test Score of 73 on his MABC assessment, ranking him in the 37^{th} percentile. A minimal detectable change (MDC) for the MABC has been reported as 1.21 points, representing a true change in motor function. The results in this child show an increase of 5 points over 5 months, which is greater than the minimal important difference (MID) of the MABC, which has been reported as a change of 2.5 points shown over 6 months. Overall, according to his occupational therapist, the patient presented with less fatigue on reassessment and was able to complete the full assessment, which he had initially been unable to accomplish. After five months of ERT, he also demonstrated an improvement in his visual motor, fine motor, and gross motor skills as measured by the Miller Function and Participation Scales (M-FUN) when comparing his initial assessment scores taken in his first month of ERT reinitiation to his reassessment scores five months later as shown in Table 4 below.

TABLE 4
Motor Improvements
Movement ABC (Manual Dexterity,
Ball Skills, & Static and Dynamic Balance)

| | Initial Assessment | Re-assessment (5 |
|---|---|---|
| | (Pre-ERT) | months on ERT |
| Total Score | 68 | 73 |
| Percentile | 25^{th} percentile | 37^{th} percentile |

Increase of 5 points with percentile increase of 12.
(minimal detectable change (MDC) = 0.28 points,
minimal important difference (MID) = 2.36 to 2.50.)

Miller Function and Participation Scales (M-FUN) )

| | Initial Assessment | Reassessment (5 months |
|---|---|---|
| | (Pre-ERT) | post ERT) |
| Visual Motor | | |
| Score* | | |
| Scaled Score | 6 (considered mild or | 9 (considered “average” or |
| | borderline delay, >1 | within 1 standard deviation |
| | standard deviation below | of the mean) |
| | the mean, but <2 standard | |
| | deviations below the mean) | |
| Progress Score | 374 | 496 |
| Assessment | Patient's scaled scores show a visual motor improvement | |
| Notes | from mild/borderline delay to the average range for his | |
| | age. His progress score is indicative of learning new motor | |
| | skills or improving his current skills. | |
| Fine | | |
| Motor Score* | | |
| Scaled Score | 3 (considered “very low or | 5 (considered mild/ |
| | severe” delay, >2 standard | borderline delay, >1 |
| | deviations below the mean) | standard deviation below |
| | | the mean, but <2 standard |
| | | deviations below the mean) |
| Progress Score | 292 | 394 |
| Assessment | Patient's scaled scores show a fine motor improvement | |
| Notes | from very low/severe fine motor delay of >2 standard | |
| | deviations below the mean to mild/borderline fine motor | |
| | delay >1 but <2 standard deviations below the mean. | |
| | Patient fatigued and | Patient was eager to |
| | required maximal | participate. He was able to |
| | encouragement to continue | participate without |
| | testing. As a result, he | excessive encouragement |
| | struggled to persevere and | on the fine motor tasks. |
| | to recruit sufficient energy | |
| | for stability and strength | |
| | tasks. | |
| Gross | | |
| Motor Score* | | |
| Scaled Score | 3 (considered very | 3 (considered very |
| | low/severe delay, >2 | low/severe delay, >2 |
| | standard deviations below | standard deviations below |
| | the mean) | the mean) |
| Progress Score | 288 | 407 |

| | |
|---|---|
| Reassessment | Patient's scaled scores indicate that his rank relative to age |
| Notes | level peers has not changed and remains very low/ |
| | severely delayed but his progress score indicates that he is |
| | gaining new motor skills and is improving in his current |
| | skills (could not hop but now can, could not adequately |
| | participate in gross motor tasks during the initial |
| | assessment but on reassessment had sufficient energy to |
| | attempt many of the tasks). |
| Overall | Patient presented with less fatigue on reassessment and |
| Assessment | was able to complete the full assessment which he was not |
| | able to on initial assessment. He demonstrated an |
| | improvement in his visual motor and fine motor skills, and |
| | although his scaled score on a gross motor level remained |
| | the same, he was also progressing on a gross motor level. |

The overall assessment findings at five months indicated that he presented with less fatigue and was able to complete the full assessment which he was not able to on initial assessment. These findings have led the patient's physicians to recommend the continuation of the infusions to treat his alglucosidase alfa responsive cytoplasmic GSD caused by a mutation in PRKAG2 gene. The patient continues on ERT at the age of 6 years (about 2 years on ERT). Per parental report, he continues to have gross motor gains but some fine motor fatigue with hand writing for longer periods of time and hypotonia.

Due to similar symptomatic phenotypes, rare PRKAG2 cases can be misdiagnosed with infantile Pompe disease. PRKAG2 should be in the differential diagnosis of cases with cardiomyopathy. Interestingly, the patient only exhibited mild cardiac hypertrophy, not typical of patients diagnosed with PRKAG2 as shown in Table 3. He did have a family member die of a sudden cardiac event, and based on current literature, there is a broader cardiac clinical spectrum of this disorder beyond cardiac involvement which includes myalgia, myopathy and seizures. The patient was diagnosed with a pathogenic mutation in PRKAG2, Gly100Ser. Other patients exhibiting the same PRKAG2 Gly100Ser mutation have been reported to have a variable cardiac presentation including ventricular pre-excitation, progressive conduction system disease, and ventricular hypertrophy. The family in the Zhang et al (2013) paper did not have muscle symptoms reported as documented in our patient. However, muscle symptoms have been reported in patients with PRKAG2; 7 of 40 patients (15%) with an N4881 mutation in PRKAG2 had myalgia/myopathy. Four patients from this cohort of 45 also had epilepsy (generalized tonic-clonic seizures), poorly controlled with medications, including 3 who also had myalgia. The present example serves to add to the phenotypic spectrum of PRKAG2 as well as highlight the importance of confirming a diagnosis of Pompe disease by more than one method. The blood based assay to diagnose Pompe disease should be performed in a lab with experience because if not done correctly, the test can result in an incorrect initial diagnosis, as noted in this case. PRKAG2 syndrome should be considered in differential diagnosis of Pompe disease. There have been two additional cases of PRKAG2 syndrome where the patients were initially clinically misdiagnosed with Pompe disease due to significant hypertrophic cardiomyopathy at presentation in one case and muscle weakness in the other (PSK personal communication).

As evidenced by the example depicting significant musculoskeletal improvements with alglucosidase alfa, a subsequent decline when ERT was withdrawn, and then improvement following reinitation of ERT, there seem to be implications of the effectiveness of alglucosidase alfa therapy for PRKAG2 deficiency. In the past, the diagnosis of Pompe disease was confirmed using GAA enzyme measurements in cultured fibroblasts or muscle cells. Enzyme measurement using acarbose, an inhibitor of alpha-glucosidase, can greatly improve the sensitivity and specificity of Pompe disease diagnosis in blood and has now been adapted in many labs as a rapid way to diagnose Pompe disease; however, without the addition of acarbose, there can be false positive results. Thus, the test needs to be done in labs with experience and expertise. It is important to broaden the diagnostic measures to include additional tests outside of enzyme testing in dried blood spots (DBS) such as gene sequencing and measurement of GAA activity in other tissue such as skin and muscle prior to initiation of ERT.

Among the GSDs, Pompe disease is the only exception with glycogen accumulation in lysosomes (lysosomal GSD) whereas all others have glycogen storage in the cytoplasm (cytoplasmic GSD). Furthermore, the use of ERT with alglucosidase alfa depends upon the mannose 6-phosphate receptor mediated enzyme uptake into lysosomes, which has been effective in reducing lysosomal glycogen storage in Pompe disease. Our group has demonstrated that ERT significantly reduced glycogen levels in the cultured primary myoblasts from skeletal muscle biopsies of patients with GSD III, a cytoplasmic GSD caused by the deficiency of glycogen debranching enzyme that leads to accumulation of abnormally structured cytoplasmic glycogen in liver and muscle. It is believed that administration of recombinant human acid alfa glucosidase enhanced lysosomal glycogen depletion, facilitated glycogen transport from the cytoplasm into lysosomes, and ultimately reduced cytoplasmic glycogen accumulation in the GSD III patient cells. As evidenced by the outcomes of this example depicting significant musculoskeletal improvements with alglucosidase alfa, a subsequent decline when ERT was withdrawn, and then improvement following reinitiation of ERT, there seem to be implications of the effectiveness of alglucosidase alfa therapy for PRKAG2 syndrome. It is possible that physical therapy and endurance exercise could be adding to the improvement in strength in our patient. However, this patient continued PT throughout his clinical course, even when ERT was discontinued with no clinical impact. It is also possible that being a carrier for Pompe disease which resulted in a decrease in residual endogenous GAA activity, could have resulted in an even greater clinical benefit from the administration of recombinant human GAA (ERT) in this case. However, in the preclinical work with GSD III, even with normal GAA activity, a benefit in cytoplasmic glycogen clearance was noted. Thus, the benefit is expected, even if the patient was not a carrier for Pompe disease.

### Example 11

### PRKAG2 Mutations Presenting in Infancy—A Possible Therapeutic Approach using Alglucosidase Alfa Enzyme Replacement Therapy

Background: PRKAG2 encodes the γ2 subunit of AMP-activated protein kinase (AMPK) which is an important regulator of cardiac metabolism. Mutations in PRKAG2 cause a cardiac syndrome comprised of ventricular hypertrophy, preexcitation, and progressive conduction system disease. Significant variability exists in the presentation and outcomes of patients with PRKAG2 mutations. The features often resemble the cardiac manifestations of Pompe disease.

Methods: Here, we add three cases to the five previously described where patients with PRKAG2 mutations presented with symptoms in infancy. In all three of our cases, Pompe disease was the initial suspected diagnosis, with two patients going on to receive enzyme replacement therapy (ERT). However, Pompe disease was eventually ruled out, and a disease causing PRKAG2 mutation was identified subsequently in each case. In one case, ERT was stopped after the PRKAG2 mutation was identified. As the motor deficits progressed on standardized measures, the treating physicians restarted ERT, and a clinical benefit was noted.

Discussion: We highlight the potential for PRKAG2 mutations to mimic Pompe disease in infancy and the need for confirmatory testing via sequencing when diagnosing Pompe disease. Also, we outline the benefit a patient showed while on ERT treatment, the decline in his condition when the infusions were discontinued, and the significant positive response when ERT was reinitiated. This further supports the role of ERT in clearing cytoplasmic glycogen.

Although the present invention has been described in considerable detail with reference to certain preferred embodiments thereof, other versions are possible. Therefore the spirit and scope of the appended claims should not be limited to the description and the preferred versions contained within this specification.

1. -19. (canceled)

20. A method of treating cytoplasmic glycogen storage disorder in an individual in need thereof comprising administering to the individual a therapeutically effective amount of acid alpha-glucosidase, wherein the acid alpha-glucosidase is administered at a first higher therapeutically effective dose weekly until a desired response is reached and then acid alpha-glucosidase is administered at a second lower therapeutically effective dose at a regular interval.

21. The method of claim 20, wherein the first higher therapeutically effective dose is about 40 mg/kg to about 100 mg/kg.

22. The method of claim 20, wherein the second lower therapeutically effective dose is about 20 mg/kg to about 80 mg/kg.

23. The method of claim 20, wherein the regular interval is selected from bimonthly, monthly, biweekly, weekly, twice weekly, daily, twice a day, three times a day, or more often a day.

24. The method of claim 20, further comprising administering to the individual an immune modulation therapy to prevent anti-acid alpha-glucosidase antibodies and infusion-associated reactions.

25. The method of claim 20, wherein the individual does not have a significant amount of fibrosis.

26. The method of claim 20, wherein the cytoplasmic glycogen storage disorder is selected from glycogen storage disease type I (GSD I), glycogen storage disease III (GSD III), glycogen storage disease IV (GSD IV), glycogen storage disease V (GSD V), glycogen storage disease VI (GSD VI), glycogen storage disease VII (GSD VII), glycogen storage disease IX (GSD IX), glycogen storage disease XI (GSD XI), glycogen storage disease XII (GSD XII), glycogen storage disease XIII (GSD XIII), glycogen storage disease XIV (GSD XIV) (phosphoglucomutase deficiency), Danon disease (GSD 2B, LAMP -2 deficiency), Lafora disease, conditions associated with a protein kinase gamma subunit 2-deficiency (PRKAG2), any other condition where there is cytoplasmic accumulation of glycogen, or a combination thereof.

27. The method of claim 20, wherein the acid alpha-glucosidase is administered as a protein, a gene therapy, or a combination thereof.

28. The method of claim 20, wherein the acid alpha-glucosidase is selected from GAA, rhGAA, neo-rhGAA, reveglucosidase alpha, an rhGAA with higher M6P content than naturally occurring GAA, a functional equivalent thereof, a portion thereof, or a combination thereof.

29. The method of claim 20, wherein the cytoplasmic glycogen storage disorder is a condition associated with PRKAG2 deficiency.

30. The method of claim 29, wherein the condition is selected from hypotonia, cardiomyopathy, cardiac hypertrophy, myopathy, cytoplasmic glycogen accumulation, ventricular hypertrophy, severe infantile hypertrophic cardiomyopathy, heart rhythm disturbances, increased left ventricular wall thickness, ventricular pre-excitation, or a combination thereof.

31. The method of claim 29, wherein the PRKAG2 deficiency is due to a mutation selected from PRKAG2 Het R531Qh mutation, PRKAG2 R302G mutation, PRKAG2 T400N mutation, PRKAG2 N4881 missense mutation, PRKAG2 R531G missense mutation, PRKAG2 G100S missense mutation, or a combination thereof.

32. The method of claim 20, wherein the cytoplasmic glycogen storage disorder is glycogen storage disease III (GSD III).

33. The method of claim 20, wherein the cytoplasmic glycogen storage disorder is glycogen storage disease IV (GSD IV).

34. The method of claim 20, wherein the cytoplasmic glycogen storage disorder is glycogen storage disease I (GSD I).
